(19) United States
(12) Patent Application Publication
(10) Pub. No.: US 2024/0409870 A1
(43) Pub. Date: Dec. 12, 2024

(54) MICROCHANNEL-BASED METHOD FOR OBTAINING THE MECHANICAL MODULUS OF A CELL, JET STREAM CONTROL METHOD, APPARATUS, ELECTRONIC DEVICE, AND STORAGE MEDIUM

(71) Applicant: Shanghai University, Shanghai (CN)

(72) Inventors: XINYUE LIU, Shanghai (CN); Jinbo Wu, Shanghai (CN); Jiajie Gong, Shanghai (CN); Xinghua Gao, Shanghai (CN); Mengying Zhang, Shanghai (CN); Guohui Hu, Shanghai (CN)

(21) Appl. No.: 18/733,925

(22) Filed: Jun. 5, 2024

(30) Foreign Application Priority Data
Jun. 9, 2023 (CN) 2023106874179
Jun. 16, 2023 (CN) 2023107220285
Dec. 21, 2023 (CN) 2023117783099

(51) Int. Cl. C12M 1/42; C12M 1/00; C12M 1/12; C12M 3/06; G06N 5/022

(52) U.S. Cl. CPC C12M 35/04; C12M 23/16; C12M 25/02; C12M 29/06; G06N 5/022

(57) ABSTRACT

The present disclosure relates to a microchannel-based method for obtaining the mechanical modulus of a cell, a microchannel-based jet stream control method, an apparatus, an electronic device, and a storage medium. The microchannel-based jet stream control method includes: obtaining the mechanical modulus of a cell before the cell enters a jet stream region of a microchannel; and adjusting the intensity of a jet stream originated from a jet stream apparatus and used to perforate the cell based on the mechanical modulus of the cell, so that the cell membrane tension of the cell is between a perforation threshold and a rupture threshold. According to the technical solution of the present disclosure, the cell transduction rate, the cell throughput, and the cell survival rate can be improved.

### CROSS-REFERENCE TO RELATED APPLICATION

This application is based upon and claims priority from. CN2023106874179 filed on 9 Jun. 2023, CN2023107220285 filed on 16 Jun. 2023, and CN2023117783099 filed on 21 Dec. 2023 the entireties of all of which are incorporated herein by reference for all purposes.

### TECHNICAL FIELD

The present disclosure relates to the field of microfluidic biotechnology, and specifically to a microchannel-based method for obtaining the mechanical modulus of a cell, a microchannel-based jet stream control method, an apparatus, an electronic device, and a storage medium.

### BACKGROUND

In a related technology, delivering exogenous macromolecules into a cell is of critical importance in basic biology and clinical applications. However, as a natural biological barrier, a cell membrane strictly prevents biological macromolecules from entering the cells, resulting in a very inefficient delivery of drugs or molecules into the cells. To overcome this obstacle, various methods for intracellular drug or molecule delivery have emerged in recent years, including a carrier-based biological method and a membrane disruption-based physical method.

With the development of micro-nano technology, there are more and more studies on intracellular material delivery based on membrane disruption. This method can bypass endocytosis and generate transient pores in the cell membrane by using external force. Before the cell membrane is resealed, exogenous molecules rapidly diffuse into the cells through the transient pores, so that the delivery of macromolecules is completed. The membrane disruption-based physical method currently includes extrusion based on microchannel contraction and hydroporation based on cell extrusion. Delivering a cell by the extrusion based on microchannel contraction is simple to operate. The hydroporation technology based on cell extrusion has the advantages of high throughput, high transfection efficiency, high cell activity, and the like.

In a related technology, transduction and transportation of macromolecular particles into cells through membranes are key steps in personalized precision medicine such as cancer cell therapy and regenerative medicine. In a related technology, a cell membrane jet stream perforation apparatus is proposed, in which a jet stream is provided, and cells are impacted by the jet stream to generate a flow field shearing force, so as to form one or more micropores in the cell membrane, so that macromolecules in a solution can enter the cells through the micropores.

### SUMMARY

An objective of the present disclosure is to provide a microchannel-based method for obtaining the mechanical modulus of a cell, a microchannel-based jet stream control method, an apparatus, an electronic device, and a storage medium, which can improve cell throughput, reduce damage to cells, and improve cell transduction efficiency and cell survival rate.

According to a first aspect of embodiments of the present disclosure, a microchannel-based jet stream control method is provided, which includes:
obtaining the mechanical modulus of a cell before the cell enters a jet stream region of a microchannel; and
adjusting the intensity of a jet stream originated from a jet stream apparatus and used to perforate the cell based on the mechanical modulus of the cell, so that the cell membrane tension of the cell is between a perforation threshold and a rupture threshold. The jet stream apparatus is used to provide the jet stream and positioned in the jet stream region.

In an embodiment, the microchannel has at least two different cross-sectional areas, the narrowest region of the microchannel has an inner diameter greater than a cell diameter and less than twice of the cell diameter to prevent the cell from being directly extruded by the microchannel when passing through the narrowest region, and only a single cell is allowed to pass through the narrowest region each time; the cell enters the jet stream region after passing through the narrowest region; and
the obtaining the mechanical modulus of a cell before the cell enters a jet stream region of a microchannel includes:
obtaining a first cell deformation data, where the first cell deformation data includes cell deformation characteristic data in which the cell is elastically deformed at one or more moments in the process of passing through the narrowest region; and
inputting the first cell deformation data into a trained prediction model for the mechanical modulus of a cell, so that the prediction model for the mechanical modulus of a cell outputs the mechanical modulus of the cell.

In an embodiment, the first cell deformation data further includes pressure difference information between an inlet of a variable cross-section region and an outlet of the variable cross-section region or flow information of the narrowest region, the variable cross-section region is a region between a cell inlet and the jet stream region of the microchannel, the variable cross-section region includes the narrowest region, and a cross-sectional area of a region in the variable cross-section region positioned on two sides of the narrowest region is greater than a cross-sectional area of the narrowest region;
the cell deformation characteristic data includes at least one of cell contour data, extension index, out-of-roundness, curvature ratio, relative cell size and reciprocal transit time; when the cell deformation characteristic data includes cell contour data, the cell contour data is coordinates of contour points on a cell contour in a cell image; and for each moment, the cell contour data includes first contour data of a front view and second contour data of a top view of the cell.

In an embodiment, the prediction model for the mechanical modulus of a cell is a fully connected neural network; the fully-connected neural network includes an input layer, a flattening layer, a merging layer, at least one hidden layer and an output layer which are connected in sequence;
the input layer is used to input the first cell deformation data; the flattening layer is used to flatten the first cell deformation data to obtain flattened first cell deformation data; the merging layer is used to merge the flattened first cell deformation data to obtain merged data; the merging layer is fully connected to the hidden layers, the last hidden layer in the at least one hidden layer is connected to the output layer, and the output layer is used to output the mechanical modulus of a cell.

In an embodiment, the obtaining cell deformation characteristic data includes:
photographing the cell through a camera device to obtain the cell image;
performing binarization on the cell image to obtain a binarized cell image;
performing contour extraction on the binarized cell image to obtain a cell contour;
determining a center of mass of the cell contour;
extracting contour points from the cell contour based on a specified angle interval to obtain N contour points, N=360/α, where α is the specified angle interval; and

obtaining a coordinate of each of the contour points; where the center of mass is an origin.

In an embodiment, the prediction model for the mechanical modulus of a cell is a convolutional neural network; the first cell deformation data is a cell image; and the cell image includes a front view and/or a top view of the cell.

In an embodiment, the prediction model for the mechanical modulus of a cell is trained by the following method:
performing calculation by using a specified constitutive model and the elastic modulus to obtain second cell deformation data; and
taking the second cell deformation data as training data, and training the prediction model for the mechanical modulus of a cell until the prediction model for the mechanical modulus of a cell meets a specified condition to obtain the trained prediction model for the mechanical modulus of a cell.

In an embodiment, the constitutive model considers the cell as a shell-like structural object, with viscous liquid inside and outside the cell; and the constitutive model is a Hookean model, a Mooney-Rivlin model, a Neo-Hookean model, a Skalak model or an Evans & Skalak model.

In an embodiment, the adjusting the intensity of a jet stream originated from a jet stream apparatus and used to perforate the cell based on the mechanical modulus of the cell includes:
obtaining a target jet stream parameter based on the mechanical modulus of the cell and a prestored first corresponding relationship; where the first corresponding relationship is a corresponding relationship between elastic modulus and jet stream parameter; and
adjusting the intensity of a jet stream originated from a jet stream apparatus and used to perforate the cell based on the target jet stream parameter.

In an embodiment, before the adjusting the intensity of a jet stream originated from a jet stream apparatus and used to perforate the cell based on the mechanical modulus of the cell, the method includes:
obtaining a target model identification of a constitutive model of the cell;
the adjusting the intensity of a jet stream originated from a jet stream apparatus and used to perforate the cell based on the mechanical modulus of the cell includes:
obtaining a target jet stream parameter based on the mechanical modulus of the cell, the target model identification and a prestored second corresponding relationship; where the second corresponding relationship is a corresponding relationship among the elastic modulus, the model identification and the jet stream parameter; and
adjusting the intensity of a jet stream originated from a jet stream apparatus and used to perforate the cell based on the target jet stream parameter.

In an embodiment, before the adjusting the intensity of a jet stream originated from a jet stream apparatus and used to perforate the cell based on the mechanical modulus of the cell, the method includes:
obtaining size data of the cell;
the adjusting the intensity of a jet stream originated from a jet stream apparatus and used to perforate the cell based on the mechanical modulus of the cell includes:
obtaining a target jet stream parameter based on the mechanical modulus of the cell, the size data and a prestored third corresponding relationship; where the third corresponding relationship is a corresponding relationship among the elastic modulus, the cell size and the jet stream parameter; and
adjusting the intensity of a jet stream originated from a jet stream apparatus and used to perforate the cell based on the target jet stream parameter.

In an embodiment, before the adjusting the intensity of a jet stream originated from a jet stream apparatus and used to perforate the cell based on the mechanical modulus of the cell, the method includes:
obtaining a target model identification of a constitutive model of the cell;
obtaining a size data of the cell;
the adjusting the intensity of a jet stream originated from a jet stream apparatus and used to perforate the cell based on the mechanical modulus of the cell includes:
obtaining a target jet stream parameter based on the mechanical modulus of the cell, the target model identification, the size data and a prestored fourth corresponding relationship; where the fourth corresponding relationship is a corresponding relationship among the elastic modulus, the model identification, the cell size and the jet stream parameter;
adjusting the intensity of a jet stream originated from a jet stream apparatus and used to perforate the cell based on the target jet stream parameter.

In an embodiment, the obtaining size data of the cell includes:
photographing the cell through a camera device to obtain the cell image; and
obtaining the size data of the cell based on the cell image.

In an embodiment, the obtaining size data of the cell includes:
obtaining a target category of the cell; and
obtaining the size data of the cell based on the target category and a fifth corresponding relationship, where the fifth corresponding relationship is a corresponding relationship between the cell category and the cell size.

In an embodiment, the target jet stream parameter includes a target jet stream amplitude, a target jet stream frequency, and pressure difference information between one side of the jet stream region close to a cell inlet of the microchannel and one side of a cell outlet of the microchannel.

In an embodiment, the mechanical modulus of a cell is a cell elastic modulus, a cell shear modulus, a cell bulk modulus, or a model parameter of a cell hyperelastic model.

In an embodiment, the microchannel-based jet stream control method may be applied to a magnetically responsive membrane-based microfluidic chip for intracellular material delivery, and the magnetically responsive membrane-based microfluidic chip for intracellular material delivery includes:
a chip body, where a microchannel and a cavity are arranged on a first face of the chip body, a middle part of the microchannel is communicated with the cavity through a transition region, and a necking of the transition region close to the microchannel end is a jet stream nozzle;
a magnetic material film layer; and
a chip base, where the chip base is provided with a through hole, and the through hole is used to make a core tip of an external alternating current coil close to the magnetic material film layer; and where a first face of the chip body is bonded to a first surface of the magnetic material film layer to close the microchannel and the cavity, the chip base is bonded to a second surface of the magnetic material film layer, the through hole of the chip base corresponds to a position of the cavity of the chip body, and the magnetic material film layer is vibrated under the excitation of an external magnetic field, so that a medium in the cavity generates a synthetic jet stream at the jet stream nozzle, which acts on the cell flowing in the microchannel, and the cell is perforated by a momentum of the synthetic jet stream to achieve material delivery.

In an embodiment, the magnetic material film layer is a composite film prepared by mixing carbonyl iron powder and PDMS, where a mass ratio of the carbonyl iron powder to the PDMS is greater than 1.

In an embodiment, the magnetic material film layer is a PDMS micromagneton sandwich film including a PDMS top layer, a PDMS bottom layer, and a permanent magnet micromagneton layer sandwiched between the PDMS top layer and the PDMS bottom layer.

In an embodiment, the magnetic material film layer is a PDMS micromagneton sandwich film including a PDMS top layer, a PDMS bottom layer, and a micromagneton layer prepared by mixing carbonyl iron powder and PDMS sandwiched between the PDMS top layer and the PDMS bottom layer.

In an embodiment, the magnetic material film layer has an amplitude not less than 5 μm.

In an embodiment, the magnetic material film layer has a thickness of 0.1˜2 mm.

In an embodiment, the magnetic material film layer has a size not less than 5×5 mm.

In an embodiment, the cavity has a diameter not less than 2 mm, the microchannel has a width not less than 20 μm, and the cavity has a height greater than that of the microchannel.

In an embodiment, the transition region is in a triangle-like shape, the base of the triangle-like shape is connected to the cavity, the apex of the triangle-like shape is connected to the microchannel, and the apex of the triangle-like shape is the jet stream nozzle.

In an embodiment, a ratio of a length of the base of the triangle-like shape to an opening width of the apex is not less than 3:1.

In an embodiment, the chip body further includes a culture medium inlet, a cell inlet and a cell outlet penetrating through a second face of the chip body, the culture medium inlet and the transition region are positioned at two opposite sides of the cavity, the cell inlet and the cell outlet are respectively positioned at two ends of the microchannel, and directions in which the cell inlet and the cell outlet penetrate through the second face of the chip body are at an obtuse angle relative to a direction in which the microchannel extends.

According to a second aspect of embodiments of the present disclosure, an electronic device is provided, which includes a memory and a processor, where the memory is configured to store a computer program executable by the processor, and the processor is configured to execute the computer program in the memory to implement the method described above.

According to a third aspect of embodiments of the present disclosure, a computer-readable storage medium is provided, which has a computer program stored thereon, and when the executable computer program in the storage medium is executed by a processor, the method described above can be implemented.

According to a fourth aspect of embodiments of the present disclosure, a computer program product is provided, which includes a computer program, and when the computer program is executed by a processor, the method described above is performed.

Compared with the prior art, the present disclosure has the beneficial effects that: according to the microchannel-based jet stream control method provided by the present disclosure, before the cell enters the jet stream region of the microchannel, the mechanical modulus of a cell is obtained, and then the jet stream intensity of the jet stream apparatus for perforating the cell is adjusted based on the mechanical modulus of the cell, so that the cell membrane tension of the cell is between the perforation threshold and the rupture threshold. In this way, insufficient cell perforation and cell rupture can be avoided, and the cell can be perforated fully, which not only improves the cell transduction efficiency, but also increases the cell survival rate.

According to the microchannel-based method for obtaining the mechanical modulus of a cell provided by the present disclosure, the first cell deformation data is obtained and is input into the trained prediction model for the mechanical modulus of a cell, so that the prediction model for the mechanical modulus of a cell outputs the mechanical modulus of the cell, where the first cell deformation data includes cell deformation characteristic data in which the cell is elastically deformed at one or more moments in the process of passing through the narrowest region of the microchannel, the microchannel has at least two different cross-sectional areas, the narrowest region of the microchannel has an inner diameter greater than a cell diameter and less than twice of the cell diameter to prevent the cell from being directly extruded by the microchannel when passing through the microchannel, and only a single cell is allowed to pass through the narrowest region each time. In this way, this method is faster than a method for measuring the mechanical modulus of a cell by using a measuring tool in a laboratory, and has high throughput, and since no measuring tool is used to apply force to the cells, the cells can be prevented from being damaged. Therefore, the technical solution provided by the present disclosure can improve the cell throughput and reduce the damage to the cells.

According to the magnetically responsive membrane-based microfluidic chip for intracellular material delivery provided by the present disclosure, the magnetically responsive membrane layer is integrated into the microfluidic chip, and the synthetic jet stream with controllable frequency is output under the excitation of an alternating magnetic field, so that the synthetic jet stream effect is applied to cells flowing in a single direction in a microchannel of the microfluidic chip, the cells are extruded and deformed, and the delivery of biological macromolecules without damage is achieved.

According to the magnetically responsive membrane-based microfluidic chip for intracellular material delivery provided by the present disclosure, a method of delivering materials to cells through synthetic jets, on the basis of ensuring high throughput, achieves control of the jet stream momentum by adjusting the parameter of the synthetic jet, thereby achieving precise control of cell membrane perforation. Compared with a hydroporation technology, this method is more flexible and universal, more efficient, and more precise and controllable.

According to the magnetically responsive membrane-based microfluidic chip for intracellular material delivery provided by the present disclosure, the momentum of the synthetic jet stream is used to perforate the cells to achieve exogenous material delivery, so that channel blockage which cannot be prevented by the hydroporation technology is fundamentally avoided, excessive flow velocity is not required, and the requirement on pressure in the channels of the microfluidic chip is low.

The microfluidic chip provided by the present disclosure is simple to prepare and low in cost, and can be plugged and used compared with a chip using a hydroporation technology. In addition, this chip is not limited to the suspended cell type, and can achieve efficient exogenous material delivery to different cells only by adjusting parameter of the synthetic jet.

### BRIEF DESCRIPTION OF DRAWINGS

FIG. 1 is a schematic diagram of a structure of a magnetically responsive membrane-based microfluidic chip for intracellular material delivery according to an exemplary embodiment.

FIG. 2 shows a relationship between the amplitude and vibration frequency of composite films with different CI/PDMS mass ratios according to an exemplary embodiment.

FIG. 3 shows a relationship between the amplitude and vibration frequency of different sized composite films with a magnetic induction intensity of 100 mT and a mass ratio of CI/PDMS=2 according to an exemplary embodiment.

FIG. 4 shows a relationship between the amplitude and frequency of PDMS micromagneton sandwich films when CI-PDMS composite films with different mass ratios of micromagnetic materials are used as micromagnetons.

FIG. 5 shows a change in a position of a neodymium-iron-boron micromagneton sandwich film within 4 s at 100 Hz.

FIG. 6 is a schematic diagram of an experiment showing intracellular material delivery using a synthetic jet stream according to an exemplary embodiment.

FIG. 7 is a flowchart of a microchannel-based method for obtaining the mechanical modulus of a cell according to an exemplary embodiment.

FIG. 8 is a schematic diagram of deformation of a cell in a microchannel according to an exemplary embodiment.

FIG. 9 is a cell image of deformation of a cell at different moments according to an exemplary embodiment.

FIG. 10 is a flowchart of a microchannel-based method for obtaining the mechanical modulus of a cell according to another exemplary embodiment.

FIG. 11 is a cell image of deformation of a cell according to an exemplary embodiment.

FIG. 12 is a binarized cell image according to an exemplary embodiment.

FIG. 13 is a cell contour image according to an exemplary embodiment.

FIG. 14 is a schematic diagram of obtaining coordinates of contour points based on a cell contour image according to an exemplary embodiment.

FIG. 15 is a schematic diagram of a structure of a prediction model for the mechanical modulus of a cell according to an exemplary embodiment.

FIG. 16 is a flowchart of a method of training a prediction model for the mechanical modulus of a cell according to an exemplary embodiment.

FIG. 17 is a block diagram of a prediction apparatus for the mechanical modulus of a cell according to an exemplary embodiment.

FIG. 18 is a flowchart of a microchannel-based jet stream control method according to an exemplary embodiment.

FIG. 19 is a schematic diagram of a structure of a zero-mass jet stream-based cell introduction microfluidic system according to an exemplary embodiment.

FIG. 20 is a flowchart of a microchannel-based jet stream control method according to an exemplary embodiment.

FIG. 21 is a flowchart of a microchannel-based jet stream control method according to another exemplary embodiment.

FIG. 22 is a flowchart of a method of training a prediction model for the mechanical modulus of a cell according to an exemplary embodiment.

FIG. 23 is a flowchart of a microchannel-based jet stream control method according to another exemplary embodiment.

FIG. 24 is a flowchart of a microchannel-based jet stream control method according to another exemplary embodiment.

FIG. 25 is a flowchart of a microchannel-based jet stream control method according to another exemplary embodiment.

FIG. 26 is a flowchart of a microchannel-based jet stream control method according to another exemplary embodiment.

FIG. 27 is a block diagram of a microchannel-based jet stream control apparatus according to an exemplary embodiment.

FIG. 28 is a block diagram of a microchannel-based jet stream control apparatus according to another exemplary embodiment.

FIG. 29 is a block diagram of an electronic device according to an exemplary embodiment.

### DETAILED DESCRIPTION OF EMBODIMENTS

Unless otherwise defined, the technical terms or scientific terms used in this specification and claims shall have the ordinary meaning understood by those of ordinary skill in the art to which the present disclosure belongs. The following describes specific embodiments of the present disclosure with reference to the accompanying drawings. It should be noted that, in the specific description of these embodiments, to provide a brief and concise description, it is impossible in this specification to describe in detail all the features of the actual embodiments. Modifications and substitutions may be made in the embodiments of the present disclosure by those skilled in the art without departing from the spirit and scope of the present disclosure, and the resulting embodiments are also within the scope of the present disclosure.

In a related technology, delivering exogenous macromolecules into a cell is of critical importance in basic biology and clinical applications. However, as a natural biological barrier, a cell membrane strictly prevents biological macromolecules from entering the cells, resulting in a very inefficient delivery of drugs or molecules into the cells. To overcome this obstacle, various methods for intracellular drug or molecule delivery have emerged in recent years, including a carrier-based biological method and a membrane disruption-based physical method. The carrier-based biological method is limited to specific cell types, has low delivery efficiency, and can produce adverse immune responses or even permanent damage to cells.

With the development of micro-nano technology, there are more and more studies on intracellular material delivery based on membrane disruption. This method can bypass endocytosis and generate transient pores in the cell membrane by using external force. Before the cell membrane is resealed, exogenous molecules rapidly diffuse into the cells through the transient pores, so that the delivery of macromolecules is completed. Although this method has the advantage of good delivery efficiency, the development and application of this method are limited by channel blockage by cells, uneven cell extrusion and the like. The membrane disruption-based physical method currently includes extrusion based on microchannel contraction and hydroporation based on cell extrusion. Delivering a cell by the extrusion based on microchannel contraction is simple to operate, which however has the biggest disadvantage that channel blockage is easily caused. The hydroporation technology based on cell extrusion-based has the advantages of high throughput, high transfection efficiency, high cell activity, and the like. However, the hydroporation technology is only limited to suspended cell types at present, has uneven cell delivery, and will cause cell blockage in most microchannels. In addition, the hydroporation technology uses the momentum of fluid hedging, which requires the chip to bear larger pressure, the transfection efficiency is greatly influenced by flow velocity and Reynolds index, and the flow of internal fluid cannot be precisely controlled.

In a related technology, the measurement techniques used to measure the cell elastic modulus include an atomic force microscope, a micropipette, a magnetic tweezer, an optical tweezer, shear flow, a cell stretcher, a micropillar array, a variable cross-section cell extrusion microchannel, and the like. However, these techniques have problems such as low throughput and large damage to cells.

In a related technology, transduction and transportation of macromolecular particles into cells through membranes are key steps in personalized precision medicine such as cancer cell therapy and regenerative medicine. In a related technology, a cell membrane jet stream perforation apparatus is proposed, in which a jet stream is provided, and cells are impacted by the jet stream to generate a flow field shearing force, so as to form one or more micropores in the cell membrane, so that macromolecules in a solution can enter the cells through the micropores. When this apparatus controls a flow field shear force in an appropriate range, one or more micropores can be formed on a cell membrane, the cell damage is reduced to the maximum extent, and the transduction efficiency is improved.

Therefore, how to control a jet stream parameter of a jet stream perforation apparatus to improve the cell transduction efficiency and the cell survival rate is also a technical problem to be solved.

To solve the technical problem described above, the present disclosure provides a magnetically responsive membrane-based microfluidic chip for intracellular material delivery, a microchannel-based method for obtaining the mechanical modulus of a cell, a microchannel-based jet stream control method, an apparatus, an electronic device, and a storage medium.

For ease of understanding, the magnetically responsive membrane-based microfluidic chip for intracellular material delivery is firstly described below, then the microchannel-based method for obtaining the mechanical modulus of a cell is described, and finally the microchannel-based jet stream control method is described.

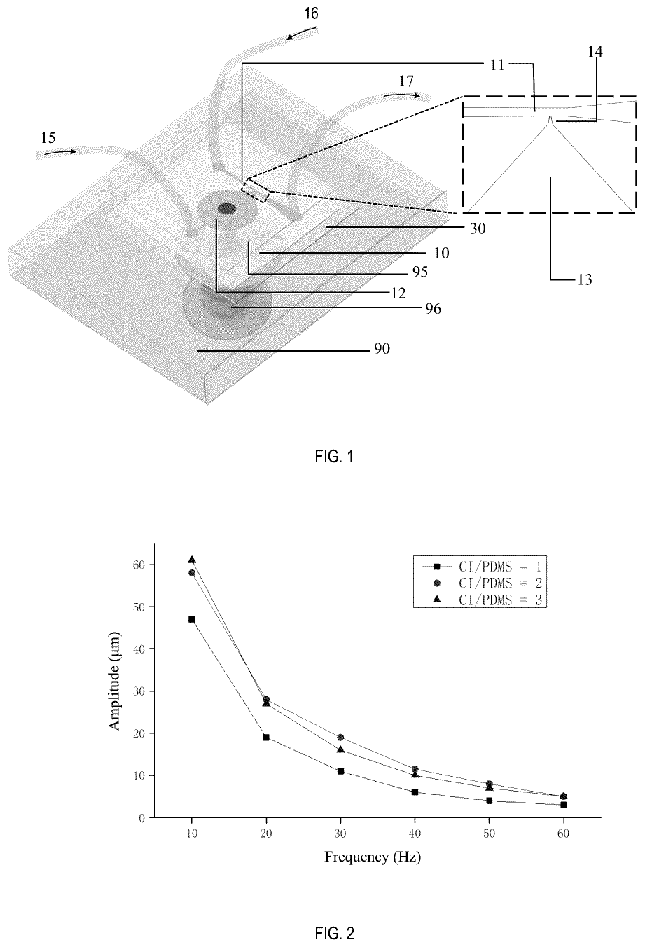

An embodiment of the present disclosure provides a magnetically responsive membrane-based microfluidic chip for intracellular material delivery. FIG. 1 is a schematic diagram of a structure of a magnetically responsive membrane-based microfluidic chip for intracellular material delivery according to an embodiment of the present disclosure. As shown in FIG. 1, the microfluidic chip includes: a chip body 10, a chip base 90 and a magnetic material film layer 30 arranged between the chip body 10 and the chip base 90. One face of the chip body 10 is provided with a microchannel 11 and a cavity 12, the microchannel 11 and the cavity 12 are equivalent to an open groove structure, the microchannel 11 is communicated with the cavity 12 through a transition region 13, where a necking of the transition region 13 close to one end of the microchannel 11 is a jet stream nozzle 14, and a dashed-line box on a right side of the FIG. 1 is an enlarged view of the transition region 13. The chip base 90 is provided with a through hole 95, and the through hole 95 is used to make a core tip of an external alternating current coil 96 close to the magnetic material film layer 30.

In this embodiment, the chip body 10 and the chip base 90 are both PDMS (polydimethylsiloxane)-based chips. One first face (a lower surface) of the chip body 10 is bonded to a first surface of the magnetic material film layer 30 to close the microchannel 11 and the cavity 12 into a closed space. The chip base 90 is bonded to a second surface of the magnetic material film layer 30. For example, in this embodiment, bonding is achieved by a plasma machine. It should be noted that the through hole 95 of the chip base 90 corresponds to a position of the cavity 12 of the chip base 90 in a vertical position, so that the core tip of the external coil exactly corresponds to the cavity position. When a controllable alternating magnetic field is applied, the magnetic material film layer 30 is vibrated under the excitation of the external magnetic field, so that a medium in the cavity 12 generates a synthetic jet stream at the jet stream nozzle 14, the synthetic jet stream acts on the cell flowing through the microchannel 11, and the cell is perforated by the momentum of the synthetic jet stream to achieve material delivery.

In this embodiment, the cavity 12 is used to store a medium, and may have a circular or approximately circular shape with a diameter of not less than 2 mm; the microchannel 11 is an elongated channel having a width of not less than 20 μm; and the height of the cavity 12 can be greater than that of the microchannel 11, for example, the height of the cavity is 80 μm to 140 μm, and the height of the microchannel 11 can be 60 μm to 120 μm. A ratio of a volume of the cavity 12 to that of the microchannel 11 is 6.3:1, so that the cavity 12 is equivalent to a liquid storage tank relative to the microchannel 11.

In this embodiment, as shown in FIG. 1, the transition region 13 is in a triangle-like shape, the base of the triangle-like shape is connected to the cavity 12, the apex of the triangle-like shape is connected to the microchannel 11, the apex of the triangle-like shape is the jet stream nozzle 14, the jet stream nozzle 14 and the microchannel 11 are in a perpendicular relationship on the same horizontal plane, and a ratio of a length of the base of the triangle-like shape to the opening width of the vertex is not less than 3:1. The purpose of this design is to provide a buffer transition zone for the jet, which prevents excessive attenuation of the jet stream velocity due to over-high instantaneous pressure and over-small jet stream nozzle size, thereby reducing the energy loss.

In this embodiment, as shown in FIG. 1, the chip body 10 further includes a culture medium inlet 15, a cell inlet 16 and a cell outlet 17 penetrating through a second face (an upper surface) of the chip body 10, the culture medium inlet 15 and the transition region 13 are positioned at two opposite sides of the cavity 12, the cell inlet 16 and the cell outlet 17 are respectively positioned at two ends of the microchannel 11, and directions in which the cell inlet 16 and the cell outlet 17 penetrate through the upper surface of the chip body 10 are at an obtuse angle relative to a direction in which the microchannel 11 extends, that is, the through holes of the cell inlet 16 and the cell outlet 17 are drilled obliquely downward, so that when the chip is used to introducing and discharging cells, the cells do not pass through a curved channel less than 90°, which causes the cells to agglomerate at the bend and block the channel.

In this embodiment, the magnetic material film layer 30 has a thickness of 100 μm, and the magnetic material film layer 30 has a size of 15 mm×15 mm. In an optional embodiment, the magnetic material film layer 30 is a composite film prepared by mixing carbonyl iron powders (CI) and PDMS, where a mass ratio of the carbonyl iron powders to the PDMS is greater than 1. A specific preparation method is as follows:
Step 1: preparing the composite material. Firstly, a PDMS prepolymer is prepared, then carbonyl iron powders are added into a liquid PDMS prepolymer based on different CI: PDMS mass ratios of 1, 2, 3 and 4, and the mixture is poured into a mortar to be ground uniformly. Finally, the mixture is placed in a vacuum drying oven to degas at room temperature for 30 min to remove air bubbles in the mixture, so that the film-forming surface in the next step will be uniform and smooth.
Step 2: spin-coating and curing to form a film. A PMMA (polymethyl methacrylate) plate is spin-coated in a spin coater, wherein a mixture of PMMA and CI-PDMS has a poor adhesion, and the mixture can be easily separated from the plate after the mixture is formed into a film, and the silanization treatment is performed if the material such as a glass plate is selected. The degassed mixture of carbonyl iron powders and PDMS at room temperature is dropped to the center of the PMMA plate, with about 2 g of the liquid mixture on each PMMA plate. The PMMA plate loaded with the mixture is placed in a spin coater, and the initial rotation speed of spin coating is set to be 500 rpm, the acceleration to be 200 rpm/s and the time to be 10 s, wherein the target rotation speed is 1000-3000 rpm, the acceleration is 500 rpm/s, and the time is 30 s. The purpose of the initial lower speed is to flatten the liquid mixture on the plate. Then, the target speed is used to adjust the height or thickness of the spinning film and to make the surface of the spinning film uniform and smooth. Finally, the mixture subjected to spin coating and the PMMA plate are placed in an oven together, and continuously heated at 80° C. for 3 h to cure.

The vibration performance of the CI-PDMS composite films is further tested below. To achieve the excitation of the synthetic jet stream in the microfluidic chip, a requirement on the vibration capability of an exciter, namely a magnetic film is provided, and the amplitude of the magnetic film is required to be not less than 5 μm. An alternating magnetic field is generated by applying a sinusoidal alternating current across the coil. In this embodiment, an alternating current coil having a pure iron core with a tip at the center is used, and the coil has a height of 3 cm and an inner diameter of 8 mm. During vibration testing, the tip of the iron core is aligned to a center of a circular CI-PDMS composite film through the through hole 95 of the chip base 90, and the displacement change of the film is tested by using a laser coaxial displacement meter at the other end of the iron core.

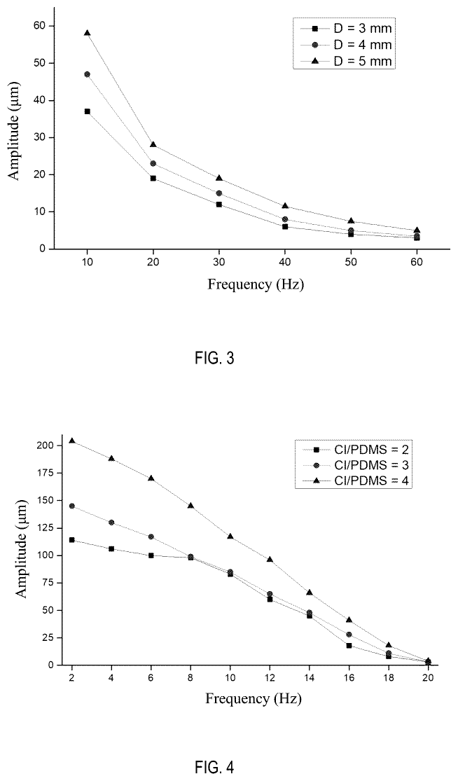

A variation of amplitude with vibration frequency of composite films with different CI/PDMS mass ratios is shown in FIG. 2. It can be observed that the amplitude of the film decreases rapidly with the increase of the vibration frequency, and the amplitude of the film with different proportions decreases to not greater than 5 μm when the frequency reaches above 60 Hz. The amplitude at a lower frequency and a high CI/PDMS mass ratio is great, and with the vibration frequency increases, the film with a CI/PDMS mass ratio equal to 2 has a better vibration effect. Based on this, the vibration effects of different sized composite films are tested when the magnetic induction intensity is 100 mT and the CI/PDMS mass ratio is 2, as shown in FIG. 3 (letter D indicates a diameter of the film). It can be found that in a range of D≤5 mm, the larger the diameter of the film is, the greater the vibration amplitude at the same frequency. With the increasing frequency to above 60 Hz, the amplitudes of the CI-PDMS composite films with various diameters are attenuated to less than 6 μm.

To obtain a magnetically responsive film that can rapidly respond to a magnetic field, has larger vibration amplitude and better vibration stability, the present disclosure further provides embedding a magnetic material in a double-layer PDMS film as a magnetic material film layer 30, and the surrounding PDMS film with a lower elastic modulus is driven by using the response of the magnetic material to the magnetic field to have a larger vibration amplitude. In a preferred embodiment, a CI-PDMS composite film with a diameter of about 500 μm and a diameter of 2 mm and a neodymium-iron-boron permanent magnet with a thickness of 1 mm and a diameter of 2 mm are respectively selected as the micromagnetons in the PDMS sandwich. The preparation process is as follows:
Step 1: preparing a bottom layer film. A PDMS prepolymer solution and a curing agent are sequentially added in a ratio of 15:1, uniformly stirred, fully mixed, and the mixture is placed in a vacuum drying oven for degassing at room temperature. The degassed PDMS is poured on a PMMA plate, and the PDMS and the PMMA plate are put into a spin coater to prepare a bottom layer film. After the spin coating is completed, the bottom layer PDMS film is placed into an oven and heating continuously at 80° C. for 30 min. The film is taken out from the oven after heating, wherein the bottom layer PDMS film is in a semi-cured state. The composite film or the micromagneton is placed on the bottom layer film, and slightly pressed to embed the bottom surface of the composite film or the micromagneton into the bottom layer PDMS film.
Step 2: preparing a top layer film. The PDMS solution is poured again above the micromagneton to completely wrap the micromagneton by the PDMS solution, and the PMMA plate is smoothly put into the spin coater. After the spin coating is completed, the entire top layer PDMS film is placed in an oven to be continuously heated for 1.5 h at the temperature of 80° C. for curing. After the heating is finished, the magnetic film with the micromagnetons sandwiched by the two PDMS films is obtained.
Step 3: taking out and fixing film. Taking the micromagneton as the center, a square film with a size not less than 5 mm×5 mm is obtained by cutting with a scalpel. This part of the film is reserved, and the rest part of the film is completely separated from the PMMA plate, which enables to smoothly take out and fix the PDMS micromagneton sandwich film. Finally, the film and the PDMS base having a hole with a diameter of 5 mm are together treaded by a plasma etching machine for 2 min. The film and the PDMS base are taken out. The center of the hole of the base is aligned with the micromagneton. The film and the micromagneton are quickly bonded. The film and the PDMS base are placed in an oven and heated at 80° C. for 8 h to ensure permanent bonding. The entire structure is removed from the PMMA plate after heating to complete the preparation of the micromagneton sandwich film. The PDMS base is obtained by reverse molding of a mold that is manufactured by 3D printing and provided with a pillar with a diameter of 5 mm and a height of 3 mm. The ratio of PDMS prepolymer solution to the curing agent is 7:1 during reverse molding. The PDMS prepolymer solution and the curing agent are heated at 70° C. for 2 h for after degassing, and the base is obtained by cutting after curing. Compared with the punched glass slide, the film removed from after being bonded to the PDMS base has a smoother appearance, firmer bonding, and no problems such as easy cracking around the holes.

The vibration performance of the PDMS micromagneton sandwich film is further tested below.

FIG. 4 shows a relationship between vibration amplitude and vibration frequency of PDMS micromagneton sandwich films when CI-PDMS composite films with different mass ratios of micromagnetic materials are used as micromagnetons, and the magnetic induction intensity is kept to be 100 mT during the test. As shown in FIG. 4, the larger the CI/PDMS mass ratio is, the better the vibrational response of the composite PDMS micromagneton sandwich film is, and the larger the amplitude is. The amplitude of this sandwich film can reach more than 100 μm within 10 Hz, but the amplitude drops rapidly with the increase of the vibration frequency. When the vibration frequency reaches 20 Hz, the membrane amplitude is only 5 μm.

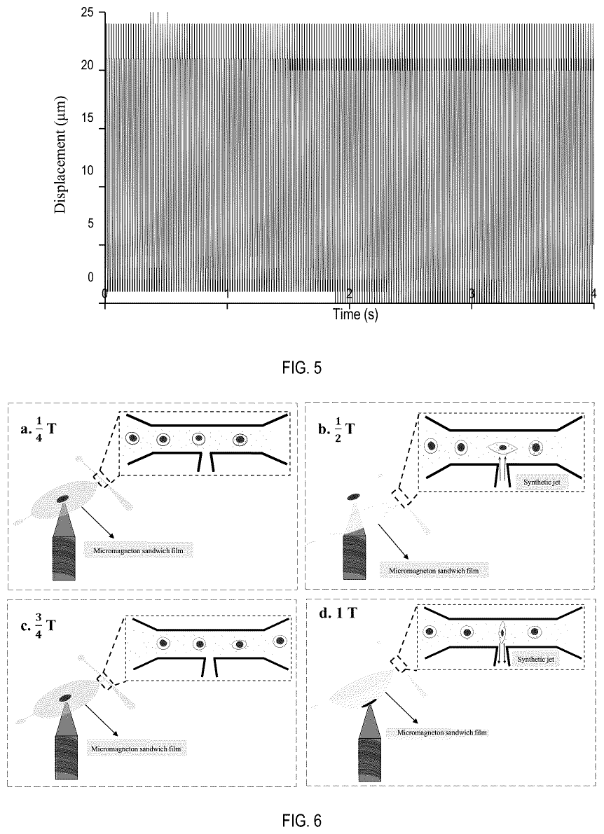

FIG. 5 shows a change in a position of a neodymium-iron-boron micromagneton sandwich film within 4 s at 100 Hz. As shown in FIG. 5, in the case where the magnetic induction intensity is 100 mT, it can be seen that the displacement amplitude, the maximum offset amount and the like all exhibit good stability. The test results of PDMS micromagneton sandwich film using neodymium-iron-boron as magnetic material indicate that a micromagneton sandwich film with a diameter of 5 mm can stably output an amplitude of 10 μm at a vibration frequency of 100 Hz in 100 mT. The experimental tests indicate that the micromagneton sandwich film can be reused multiple times, and maintains good response amplitude stability after continuous vibration for more than 30 min each time.

The method for achieving intracellular molecule delivery by using the synthetic jet stream of the magnetically responsive membrane-based microfluidic chip according to the embodiment of the present disclosure is as follows.

A culture medium is slowly injected into the chip at a speed of at most 1 μL/min through the culture medium inlet 15 until the cavity 12 and the microchannel 11 are filled with the medium, and then a silicone tube connected to the culture medium inlet 15 is clamped by a steel clamp so as to seal the culture medium inlet 15.

A cell culture medium with fluorescent molecules is prepared. 1 mL of cell suspension for experiments is obtained and supplemented with a culture medium based on a requirement of the experiment to dilute the cell suspension, and then glucan is added into the cell suspension based on a certain proportion by using a pipettor and re-suspended. The cell suspension containing dextran is sucked into a syringe. The syringe needle is connected to a silicone tube and a small steel tube, and passes through the cell inlet 16. Then, the syringe is fixed in a syringe pump. Similarly, a syringe controlled by the extraction pump and sequentially connected to a silicone tube and a small steel tube passes through the cell outlet 17 and is fixed.

The synthetic jet stream is excited and introduced into a cell. The syringes are connected to the inlet and outlet of the PDMS microfluidic chip through the small steel tubes, and the injection and extraction pumps are turned on to allow cells to pass into the microchannel 11. Meanwhile, a signal generator is turned on and alternating current voltage is applied across the coil, so that the magnetic material film layer 30 (micromagneton sandwich film) starts to vibrate, and a synthetic jet stream is generated at the jet stream nozzle 14 in the chip, which acts on the cells flowing in the microchannel 11. Then a peristaltic pump is started to perform water-cooling heat dissipation on the coil, wherein the inlet and the outlet of the peristaltic pump are connected to an ice water tank. In addition, a control test is used in the research, after cells in a control group are introduced into the microchannel of the chip, no voltage is applied to two ends of the coil, namely, no vibration is applied to the micromagneton sandwich film. The effects of synthetic jet stream cannot be generated in the microfluidic chip, and other operations and time are the same as those in an experimental group. FIG. 6 is a schematic diagram of an experiment showing intracellular material delivery using a synthetic jet stream according to an embodiment of the present disclosure. It can be seen that synthetic jet stream effect is exerted on the cells flowing in a single direction in the microchannel of the microfluidic chip, so that the cells are extruded and deformed under the momentum effect of the synthetic jet, and the delivery of biological macromolecules without damage is achieved. In addition, due to the application of an alternating magnetic field, in an alternating period (T), a direction of the synthetic jet stream changes in a forward direction (a direction from the jet stream nozzle 14 toward the microchannel 11) and a reverse direction (a direction from the microchannel 11 toward the jet stream nozzle 14). This allows cells to switch between transverse stretching and longitudinal stretching, which is more conducive to achieving extrusion deformation.

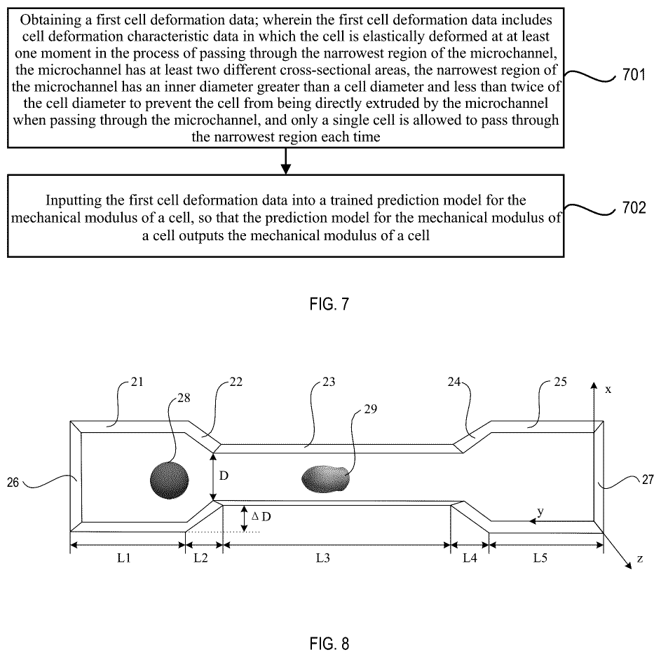

FIG. 7 is a flowchart of a microchannel-based method for obtaining the mechanical modulus of a cell according to an exemplary embodiment. The microchannel-based method for obtaining the mechanical modulus of a cell may be applied to electronic devices such as a computer and a server. Referring to FIG. 7, the microchannel-based method for obtaining the mechanical modulus of a cell may include the following steps:

Step 701: obtaining a first cell deformation data; wherein the first cell deformation data includes cell deformation characteristic data in which the cell is elastically deformed at one or more moments in the process of passing through the narrowest region of the microchannel, the microchannel has at least two different cross-sectional areas, the narrowest region of the microchannel has an inner diameter greater than a cell diameter and less than twice of the cell diameter to prevent the cell from being directly extruded by the microchannel when passing through the microchannel, and only a single cell is allowed to pass through the narrowest region each time.

In this embodiment, as shown in FIG. 8, the microchannel 11 may include a first conduit section 21, a second conduit section 22, a third conduit section 23, a fourth conduit section 24 and a fifth conduit section 25 which are connected in sequence, cross-sectional areas of the first conduit section 21, the third conduit section 23 and the fifth conduit section 25 are uniformly distributed along a length direction of the microchannel, a cross-sectional area of the first conduit section 21 is the same as that of the fifth conduit section 25, and a cross-sectional area of the first conduit section 21 is greater than a cross-sectional area of the third conduit section 23. The second conduit section 22 and the fourth conduit section 24 are transition conduit sections. A cross-sectional area of the second conduit section 22 close to the first conduit section 21 is greater than that of the second conduit section 22 close to the third conduit section 23, and a cross-sectional area of the second conduit section 22 decreases in a direction of the first conduit section 21 towards the third conduit section 23. A cross-sectional area of the fourth conduit section 24 close to the third conduit section 23 is less than that of the fourth conduit section 24 close to the fifth conduit section 25, and the cross-sectional area of the fourth conduit section 24 increases in a direction of the first conduit section 21 towards the third conduit section 23.

As shown in FIG. 8, the third conduit section 23 is the narrowest region of the microchannel 11, and the third conduit section 23 has an inner diameter D of 7 μm, which is greater than a diameter of the undeformed cell 28 and less than twice the diameter of the cell 28, so as to prevent the undeformed cell 28 from being directly extruded by the microchannel 11 when passing through the microchannel, and only a single cell is allowed to pass through the narrowest region each time. Allowing only a single cell to pass through the narrowest region each time means that only one cell is present at any one location within the narrowest region at the same moment.

As shown in FIG. 8, in an XYZ-coordinate system, the microchannel 11 extends in the y-axis direction. A length L1 of the first conduit section 21 is the same as a length L5 of the fifth conduit section 25, a length L3 of the third conduit section 23 is a sum of the length L1 of the first conduit section 21 and the length L5 of the fifth conduit section 25, and a length L2 of the second conduit section 22 is the same as a length L4 of the fourth conduit section 24.

In an embodiment, the length L1 of the first conduit section 21 is 15 micrometers, the length L2 of the second conduit section 22 is 5 micrometers, the length L3 of the third conduit section 23 is 30 micrometers, the length L4 of the fourth conduit section 24 is 5 micrometers, and the length L5 of the fifth conduit section 25 is 15 micrometers. The difference ΔD between an outer diameter of the first conduit section 21 and an outer diameter of the third conduit section 23 is 3.5 micrometers. Of course, in other embodiments, the dimensions of the portions of the microchannel 11 may also be other values.

As shown in FIG. 8, an inlet 26 of the microchannel 11 is positioned at one side of the first conduit section 21, and an outlet 27 of the microchannel 11 is positioned at one side of the fifth conduit section 25. A pressure in the microchannel 11 at the inlet 26 is greater than that at the outlet 27, so that the cells flow out from the outlet 27 after entering the microchannel 11 from the inlet 26 of the microchannel 11. After the undeformed cells 28 enter the third conduit section 23, the elastically deformed cells are extruded, and the force-sensitive channel of the elastically deformed cells 29 is opened. After the force-sensitive channel of the cell is opened, the macromolecules can be transported into the cell by using a jet stream technical means. However, to better set a control parameter of the jet stream apparatus, it is necessary to know the elastic modulus of the cells.

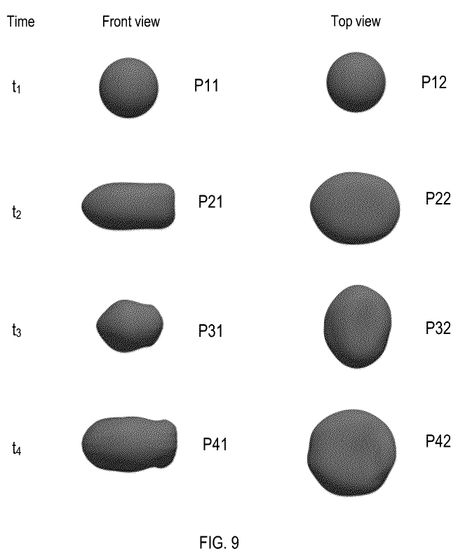

In this embodiment, as shown in FIG. 9, the cells are elastically deformed to different degrees at different moments when passing through the microchannel 11. For example, at a first momentt₁, the cell is not elastically deformed, and a front view P11 and a top view P12 of the cell are both circular; at a second moment t₂, a third moment t₃ and a fourth moment t₄, the cell is elastically deformed to varying degrees, a front view P21 and a top view P22 of the cell at the second moment t₂, a front view P31 and a top view P32 of the cell at the third moment t₃, and a front view P41 and a top view P42 of the cell at the fourth moment t₄ are all irregular.

In this embodiment, the first cell deformation data includes cell deformation characteristic data in which the cell is elastically deformed at a plurality of moments in the process of passing through the narrowest region of the microchannel 11. In other embodiments, the first cell deformation data may only include cell deformation characteristic data in which the cell is elastically deformed at a certain moment in the process of passing through the microchannel 11.

In this embodiment, the cell deformation characteristic data is cell contour data, and the first cell deformation data further includes pressure difference information between an inlet of a variable cross-section region and an outlet of the variable cross-section region or flow information of the narrowest region. The variable cross-section region is a region between a cell inlet 16 and the jet stream region of the microchannel 11, the variable cross-section region includes the narrowest region, and a cross-sectional area of a region in the variable cross-section region positioned on two sides of the narrowest region is greater than a cross-sectional area of the narrowest region. The pressure difference information and the flow information are equivalent and can be converted through a conversion formula. The cell contour data is coordinates of contour points on the cell contour in the cell image. The cell image includes a front view and a top view of the cell. For each moment, the cell contour data includes first contour data of a front view and second contour data of a top view of the cell. In other embodiments, the cell image may include only a front view or a top view of the cell.

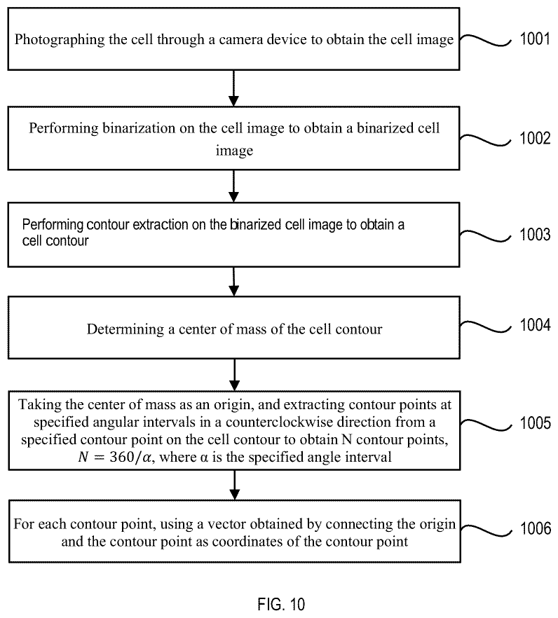

In this embodiment, as shown in FIG. 10, for each cell image, the method of obtaining cell contour data may include the following steps:
Step 1001: photographing the cell through a camera device to obtain the cell image.

In this embodiment, an example of obtaining coordinates of contour points on a cell contour in a front view of a cell is described.

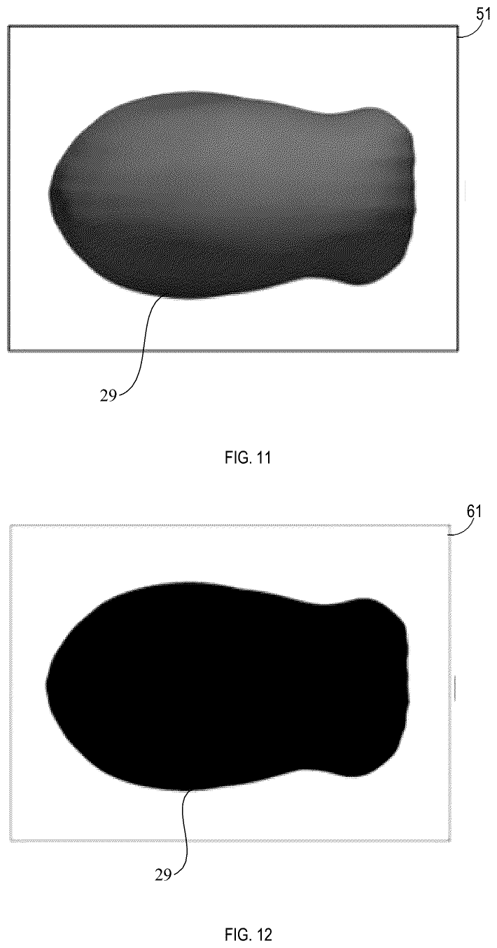

In this step, as shown in FIG. 11, a cell image 51 including an elastically deformed cell 29 can be obtained by photographing a front surface of the cell by the camera device, and the cell image 51 is a front view of the cell.

Step 1002: performing binarization on the cell image to obtain a binarized cell image.

In this step, as shown in FIG. 12, the cell image 51 shown in FIG. 11 is binarized to obtain a binarized cell image 61.

Step 1003: performing contour extraction on the binarized cell image to obtain a cell contour.

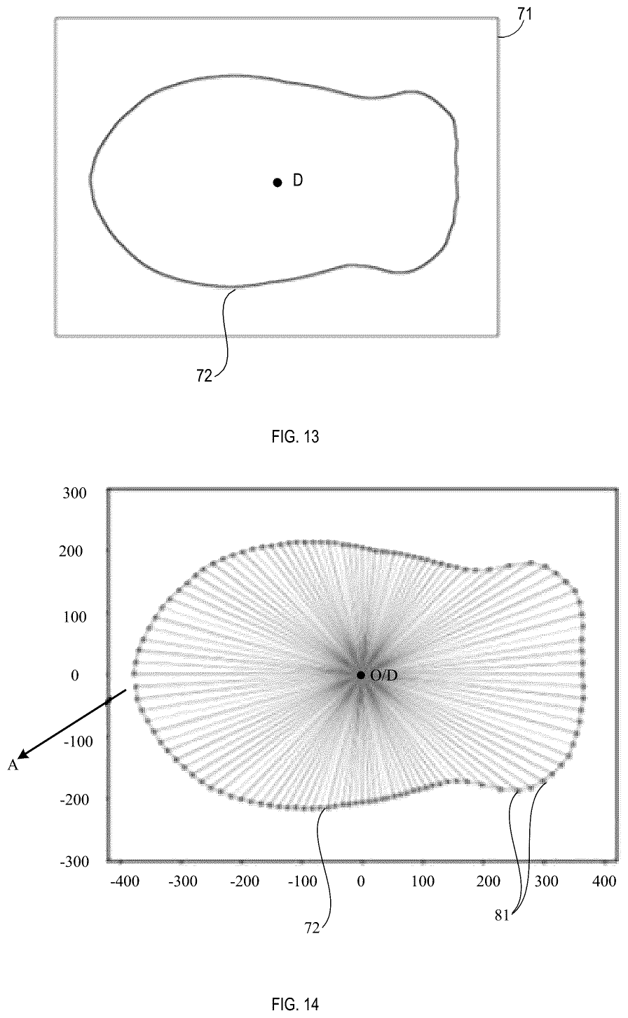

In this step, as shown in FIG. 13, the cell contour in the binarized cell image 61 may be detected by using a boundary following algorithm proposed by Suzuki et al. to obtain a cell contour image 71, and the cell contour image 71 may include a cell contour 72, or a cell contour may be extracted by using another algorithm. The cell contour is a footprint contour, not a cross-sectional contour.

Step 1004: determining a center of mass of the cell contour.

In this step, a center of mass of the cell contour 72 is calculated. As shown in FIG. 13, the center of mass D is positioned in the cell contour 72, which is also a centroid of the cell contour 72.

Step 1005: taking the center of mass as an origin, and extracting contour points at specified angular intervals in a counterclockwise direction from a specified contour point on the cell contour to obtain Ncontour points, N=360/α, where α is the specified angle interval. In this embodiment, α is 2°, and N is 180.

In this step, as shown in FIG. 14, from a specified contour point A on the cell contour 72, contour points are extracted at angular intervals of 2° in a counterclockwise direction to obtain 180 contour points.

In this embodiment, 180 contour points are taken from the cell contour 72, and these 180 contour points are sufficient to describe the cell contour 72, so that the calculation amount can be reduced, and the calculation speed can be increased. Of course, in other embodiments, other numbers of contour points may be taken.

Of course, in other embodiments, the manner of extracting contour points from the cell contour is not limited to that in this embodiment. For example, 180 contour points may be obtained by extracting contour points at angular intervals of 2° in a clockwise direction from a specified contour point A on the cell contour 72.

Step 1006: for each contour point, using a vector obtained by connecting the origin and the contour point as coordinates of the contour point.

In this step, as shown in FIG. 14, for each contour point 81, a corresponding vector is obtained by connecting the origin O and the contour point 81, and this vector is used as the coordinates of the contour point 81. A combination of these 180 vectors is an array describing the cell contour, which is also a set of input data for the prediction model for the mechanical modulus of a cell. If the array is denoted as X₁ (180, 2), then the array X₁ (180, 2) is as follows:

| | |
|---|---|
| x₁ | y₁ |
| x₂ | y₂ |
| x₃ | y₃ |
| . . . | . . . |
| x₁₈₀ | y₁₈₀ |

In an embodiment, after the coordinates of the contour point are obtained, a normalization process is required for the coordinates of the contour point, and the normalization can make the value range of the data input to the prediction model for the mechanical modulus of a cell in a relatively consistent interval. Moving the center of mass D to the origin O makes normalization easier. In addition, the normalization of the data input to the prediction model for the mechanical modulus of a cell helps to increase the convergence rate of the model, provide robustness of the model, and avoid variability between features.

The method of obtaining cell contour data is described above, and both the first contour data of the front view and the second contour data of the top view of the cell can be obtained by the above method. Details are not described herein.

Step 702: inputting the first cell deformation data into a trained prediction model for the mechanical modulus of a cell, so that the prediction model for the mechanical modulus of a cell outputs the mechanical modulus of the cell.

In this embodiment, the mechanical modulus of a cell may be a cell elastic modulus. In other embodiments, the mechanical modulus of a cell may also be a cell elastic modulus, a cell shear modulus, a cell bulk modulus, or a model parameter of a cell hyperelastic model. It should be noted that the cell elastic modulus, cell shear modulus, cell bulk modulus and the model parameter of the cell hyperelastic model are equivalent and can be converted through the conversion formula.

For example, for the cell hyperelastic model, the model parameters (such as C₁₀, C₀₁, C₁₁, C₁₂, C₂₁, C₂₀, C₀₂, C₃₀ and C₀₃ model parameters of the Mooney-Rivlin model, C₁₀, C₀₁, C₂₀, C₀₂, and C₁₁ model parameters of the Polynomial Form model, and C₁₀, C₂₀ and C₃₀ model parameters of the Yeoh Model) can be calculated by conversion formula to obtain the cell elastic modulus, the cell shear modulus or the cell bulk modulus. The conversion formula between the modulus and the model parameter is different for different constitutive models.

In this embodiment, the model selected for the cell is a cell model of a shell structure, which considers a cell membrane as a shell structure, with viscous liquid inside and outside the cell. For the cell model of the shell structure, the cell membrane constitutive model uses a Hookean model, a Mooney-Rivlin model, a Neo-Hookean model, a Skalak model or an Evans & Skalak model. For example, in this embodiment, the cell membrane constitutive model may be the Mooney-Rivlin model.

The constitutive model may also be referred to as a constitutive equation. For example, the following equation is a constitutive model, where C₁₀ and C₀₁ are elastic moduli. The elastic modulus may be referred to as a constitutive parameter.

$$U = {C}_{10} ( {J}_{1} - 3 ) + {C}_{01} ( {J}_{2} - 3 ) + \frac{κ}{2} {( {J}_{3} - 1 )}^{2}$$

where U is strain energy density, J₁=I₁I₃^{-1/3}, J₂=I₂I₃^{-2/3}, J₃=I₃^{1/2}, I₁, I₂ and I₃ are three deformation invariants of a Cauchy-Green tensor respectively, and κ is a material constant and is used to describe the nonlinear characteristic of the cell.

If the known condition is that the cell membrane constitutive model uses the above equation, the elastic modulus therein can be predicted using the prediction model for the mechanical modulus of a cell trained on the above equation.

In this embodiment, the prediction model for the mechanical modulus of a cell is used to predict the mechanical modulus of a cell for a given constitutive model. For example, if the cell membrane constitutive model uses the Mooney-Rivlin model, the prediction model for the mechanical modulus of a cell is used to predict the mechanical modulus of a cell when the cell membrane constitutive model uses the Mooney-Rivlin model.

In this step, the obtained first cell deformation data is input into the trained prediction model for the mechanical modulus of a cell, and the prediction model for the mechanical modulus of a cell outputs the mechanical modulus of the cell.

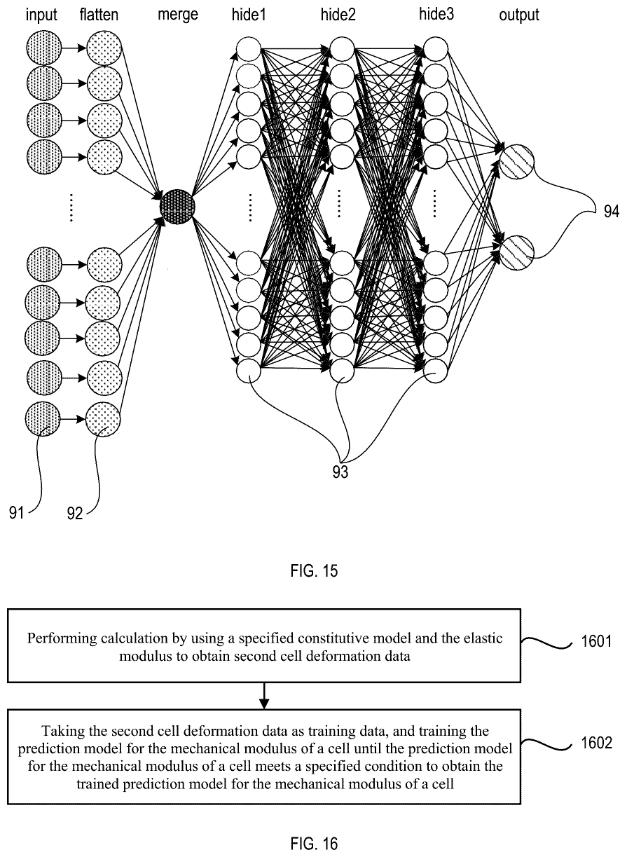

In this embodiment, as shown in FIG. 15, the prediction model for the mechanical modulus of a cell is a fully connected neural network. The fully-connected neural network includes an input layer (input), a flattening layer (flatten), a merging layer (merge), at least one hidden layer (hide1, hide2, hide3) and an output layer (output) which are connected in sequence. The number of the hidden layers may be other values and is not limited to 3.

The input layer (input) is used to input first cell deformation data. The input layer includes 2n+1 input nodes 91 for inputting cell contour data from a first instant t₁ to an n^{th} instant (tₙ) and pressure difference information between an inlet of the variable cross-section region and an outlet of the variable cross-section region. n is a positive integer. The pressure difference between the inlet of the variable cross-section region and the outlet of the variable cross-section region is a constant value, which can be denoted as an array P(1). Each input node 91 is used to input an array. For example, at the first moment t₁, the cell contour data includes first contour data of the front view of the cell (denoted as X₁ (180, 2)) and second contour data of the top view (denoted as X₂ (180, 2)); at the second moment t₂, the cell contour data includes first contour data of the front view (denoted as X₃ (180, 2)) and second contour data of the top view (denoted as X₄ (180, 2)) of the cell, . . . , and at the n^{th} moment (tₙ), the cell contour data includes first contour data of the front view (denoted as X₂ₙ₋₁ (180, 2)) and second contour data of the top view (denoted as X₂ₙ (180, 2)) of the cell. The 2n+1 input nodes 91 of the input layer are used to output the arrays X₁ (180, 2), X₂ (180, 2), X₃ (180, 2), X₄ (180, 2), . . . , X₂ₙ₋₁ (180, 2), X₂n (180, 2) and P(1). The cell contour data may be obtained by processing the cell image using a CV2 library (computer vision library) in Python.

The flattening layer (flatten) is used to flatten the first cell deformation data to obtain flattened first cell deformation data. The flattening layer (flatten) includes 2n+1 flattening nodes 92, and the 2n+1 flattening nodes 92 are connected to the 2n+1 input nodes 91 in a one-to-one correspondence manner. The flattening nodes 92 are used to perform flattening processing on the data received by the corresponding input nodes 91.

The merging layer (merge) is used to perform merging processing on the flattened first cell deformation data to obtain merged data, where the merged data is a tensor. The merging layer is connected to all flattening nodes 92 in flattening layer (flatten). In this embodiment, the flattened first cell deformation data may be concatenated using the Concatenate function to obtain the merged data. However, this is not limited thereto. The merging layer (merge) is fully connected to the hidden layers (hide1, hide2 and hide3).

The hidden layer (hide1) includes 2n+1 neuron nodes 93, and each neuron node 93 in the hidden layer (hide1) is connected to the merging layer (merge). The hidden layer (hide2) also includes 2n+1 neuron nodes 93, and each neuron node 93 in the hidden layer (hide2) is connected to each neuron node 93 in the hidden layer (hide1) and to each neuron node 93 in the hidden layer (hide3).

The hidden layer (hide3) is connected to the output layer (output), and the output layer is used to output the mechanical modulus of a cell. The output layer (output) includes output nodes 94, the number of output nodes 94 is the same as that of cell mechanical moduli, and each output node 94 is used to output one mechanical modulus of a cell. For example, when the mechanical modulus of a cell includes c₁ and c₂, the output layer (output) includes two output nodes 94, where one output node 94 is used to output C₁₀, and the other output node 94 is used to output C₀₁.

The method for predicting the mechanical modulus of a cell by using the trained prediction model for the mechanical modulus of a cell is described above. The following describes a method of training a prediction model for the mechanical modulus of a cell.

As shown in FIG. 16, the method of training a prediction model for the mechanical modulus of a cell may include the following steps:
Step 1601: performing calculation by using a specified constitutive model and the elastic modulus to obtain second cell deformation data; and
Step 1602: taking the second cell deformation data as training data, and training the prediction model for the mechanical modulus of a cell until the prediction model for the mechanical modulus of a cell meets a specified condition to obtain the trained prediction model for the mechanical modulus of a cell.

In this embodiment, a specified constitutive model and an elastic modulus may be used to perform calculation, and corresponding second cell deformation data may be obtained as training data. For example, the specific constitutive model may be the above equation, and M sets of values of C₁₀ and C₀₁ are prepared. The values of C₁₀ and C₀₁ are different in each set. For each set of values of C₁₀ and C₀₁, corresponding second cell deformation data is calculated using the above equation to describe a shape of the cell after deformation.

Then the training data obtained by calculation is input into the prediction model for the mechanical modulus of a cell, and the prediction model for the mechanical modulus of a cell is trained until the prediction model for the mechanical modulus of a cell meets the specified conditions to obtain the trained prediction model for the mechanical modulus of a cell. The specified conditions are that the training loss and the verification loss reach 10⁻⁵-10⁻⁴, and the precision of the test set reaches more than 99%.

The training data is obtained by calculation, so that massive training data can be quickly obtained, the training speed is improved, and the training cost is saved. Of course, the training data may also be experimental data.

In this embodiment, the first cell deformation data is obtained and is input into the trained prediction model for the mechanical modulus of a cell, so that the prediction model for the mechanical modulus of a cell outputs the mechanical modulus of the cell; in this way, this method is faster than a method for measuring the mechanical modulus of a cell by using a measuring tool in a laboratory, and has high cell throughput, and since no measuring tool is used to apply force to the cells, the cells can be prevented from being damaged. The cell throughput refers to the number of cells processed by the apparatus per unit time. Therefore, the technical solution provided by the present disclosure can improve the cell throughput of the apparatus and reduce the damage to the cells.

In other embodiments, the cell deformation characteristic data may further include any one or any combination of cell contour data, extension index, out-of-roundness, curvature ratio, relative cell size and reciprocal transit time.

In an embodiment, a calculation formula for the extension index is as follows:

$$D 1 = ( W - H ) / ( W + H )$$

where D1 is the extension index, W is the maximum length of the cell after deformation, and H is the minimum length of the cell after deformation.

In another embodiment, a calculation formula for the extension index is as follows:

$$D 1 = \frac{W}{H}$$

where W is the maximum length of the cell after deformation, and H is the minimum length of the cell after deformation.

In another embodiment, a calculation formula for the extension index is as follows:

$$D 1 = \frac{{D}_{t}}{{D}_{t + 1}} = \frac{{W}_{t} / {H}_{t}}{{W}_{t + 1} / {H}_{t + 1}}$$

where Dₜ is the deformation of the cell at the t moment, Dₜ₊₁ is the deformation of the cell at the t+1 moment, Wₜ is the maximum length of the cell after deformation at the t moment, Hₜ is the minimum length of the cell after deformation at the t moment, Wₜ₊₁ is the maximum length of the cell after deformation at the t+1 moment, and Hₜ₊₁ is the minimum length of the cell after deformation at the t+1 moment.

In another embodiment, a calculation formula for the extension index is as follows:

$$D 1 = W / 2 {R}_{1}$$

where R₁ is a radius of the cell without deformation, and W is the maximum length of the cell after deformation.

A calculation formula for out-of-roundness is as follows:

$$O = 1 - c \\ c = \frac{2 \sqrt{A π}}{p}$$

where O is out-of-roundness, c is roundness, A is an area of a cross-section of the cell after deformation, and p is a perimeter of the cross-section of the cell after deformation.

A calculation formula for curvature ratio is as follows:

$$ratio = {κ}_{min} / {κ}_{max}$$

where ratio is a curvature ratio, κₘᵢₙ is a minimum curvature of a cross-section of the cell after deformation, and κₘₐₓ is a maximum curvature of a cross-section of the cell after deformation.

A calculation formula for relative cell size is as follows:

$$D 2 = {R}_{1} / {R}_{2}$$

where D2 is the relative cell size, R₁ is a radius of the cell without deformation, and R₂ is a radius of an inner wall cross-section of the third conduit section 23.

A calculation formula for reciprocal transit time is as follows:

$$D 3 = \frac{1}{Passage time}$$

where D3 is the reciprocal transit time, and Passage time is a time period for the cell to pass through the third conduit section 23.

Another exemplary embodiment of the present disclosure further provides a microchannel-based method for obtaining the mechanical modulus of a cell. Unlike the embodiments described above, in this embodiment, the prediction model for the mechanical modulus of a cell is a convolutional neural network. The first cell deformation data is a cell image photographed of a deformed cell, and the cell image includes a front view and a top view of the cell.

In this embodiment, the collected cell image may be directly input into the trained prediction model for the mechanical modulus of a cell, and the prediction model for the mechanical modulus of a cell may directly output the mechanical modulus of a cell. Therefore, after the cell image is collected and before the prediction model for the mechanical modulus of a cell is input, the step of converting the cell image into data is omitted, so that the use is convenient, and the obtaining speed of the mechanical modulus of a cell can be improved.

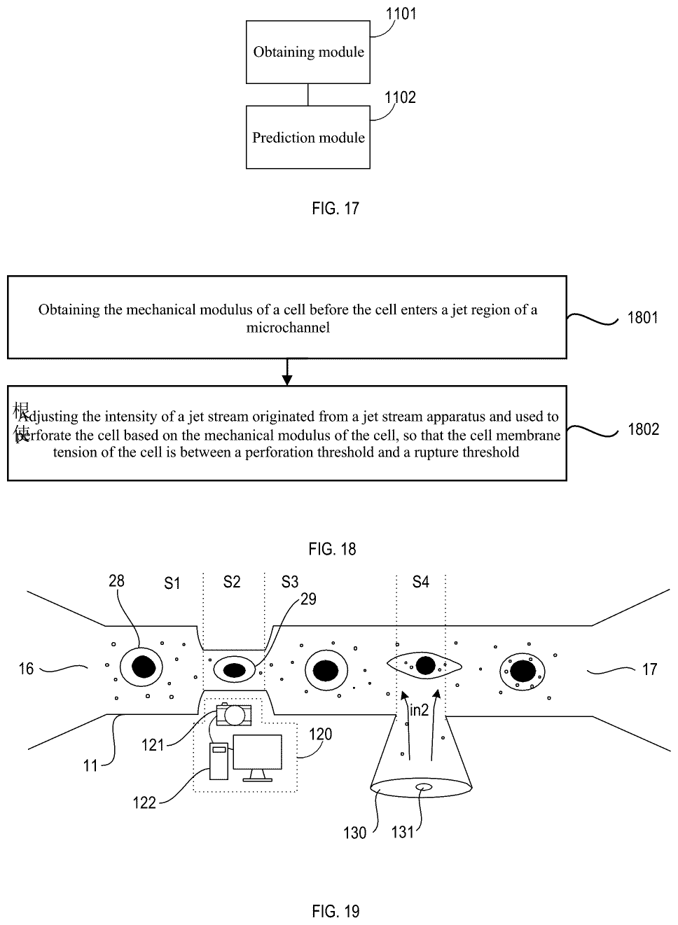

FIG. 17 is a block diagram of a microchannel-based mechanical modulus of a cell obtaining apparatus according to an exemplary embodiment. As shown in FIG. 17, in this embodiment, the microchannel-based mechanical modulus of a cell obtaining apparatus includes:
an obtaining module 1101, configured to obtain first cell deformation data; where the first cell deformation data includes cell deformation characteristic data in which the cell is elastically deformed at one or more moments in the process of passing through the narrowest region of the microchannel, the microchannel has at least two different cross-sectional areas, the narrowest region of the microchannel has an inner diameter greater than a cell diameter and less than twice of the cell diameter to prevent the cell from being directly extruded by the microchannel when passing through the microchannel, and only a single cell is allowed to pass through the narrowest region each time;
a prediction module 1102, configured to input the first cell deformation data into a trained prediction model for the mechanical modulus of a cell, so that the prediction model for the mechanical modulus of a cell outputs the mechanical modulus of the cell.

FIG. 18 is a flowchart of a microchannel-based jet stream control method according to an exemplary embodiment. Referring to FIG. 18, the microchannel-based jet stream control method may include the following steps:
Step 1801: obtaining the mechanical modulus of a cell before the cell enters a jet stream region of a microchannel; and
Step 1802: adjusting the intensity of a jet stream originated from a jet stream apparatus and used to perforate the cell based on the mechanical modulus of the cell, so that the cell membrane tension of the cell is between a perforation threshold and a rupture threshold, and the jet stream apparatus is positioned in the jet stream region of the microchannel.

The mechanical properties of the cells are different, and the optimal jet stream working conditions of the corresponding jet stream apparatus are also different. The mechanical properties of the cells and the optimal jet stream working conditions are in a one-to-one correspondence relationship. The mechanical characteristics of cells can be represented by a constitutive model and the mechanical modulus of a cell, and the jet stream working condition of the jet stream apparatus can be represented by the jet stream intensity. Therefore, when the constitutive model of the cell is known, there is a one-to-one correspondence relationship between the mechanical modulus of a cell and the optimal jet stream intensity. Furthermore, the jet stream intensity of the jet stream apparatus can be adjusted based on the mechanical modulus of the cell.

In this embodiment, before the cell enters the jet stream region of the microchannel, the mechanical modulus of a cell may be obtained through an experimental means, and of course, the mechanical modulus of a cell may also be obtained through other technical means.

In this embodiment, the mechanical modulus of a cell may be a cell elastic modulus. In other embodiments, the mechanical modulus of a cell may also be a cell elastic modulus, a cell shear modulus, a cell bulk modulus, or a model parameter of a cell hyperelastic model. It should be noted that the cell elastic modulus, cell shear modulus, cell bulk modulus and the model parameter of the cell hyperelastic model are equivalent and can be converted through the conversion formula.

In this embodiment, before the cell enters the jet stream region of the microchannel, the mechanical modulus of a cell is obtained, and then the jet stream intensity of the jet stream apparatus for perforating the cell is adjusted based on the mechanical modulus of the cell, so that the cell membrane tension of the cell is between the perforation threshold and the rupture threshold. In this way, insufficient cell perforation and cell rupture can be avoided, and the cell can be perforated fully, which not only improves the cell transduction efficiency, but also increases the cell survival rate.

The above briefly describes the microchannel-based jet stream control method provided by the present disclosure, and the microchannel-based jet stream control method is described in detail below.

The microchannel-based jet stream control method may be applied to a zero-mass jet stream-based cell introduction microfluidic system. Before describing the microchannel-based jet stream control method in detail, the zero-mass jet stream-based cell introduction microfluidic system is firstly described.

As shown in FIG. 19, the zero-mass jet stream-based cell introduction microfluidic system includes a microchannel 11, a mechanical monitoring device 120 and a jet stream apparatus 130. The microchannel 11, the mechanical monitoring device 120 and the jet stream apparatus 130 can be assembled to form a zero-mass jet stream-based cell introduction microfluidic system.

As shown in FIG. 19, the microchannel 11 is a variable cross-section conduit and has at least two different cross-sectional areas. The microchannel 11 includes a cell inlet 16, a first section S1, a second section S2, a third section S3 and a fourth section S4 connected in sequence, a jet stream inlet in2, and a cell outlet 17. The cross-sectional areas of the first section S1, the third section S3 and the fourth section S4 are greater than that of the second section S2, and the second section S2 is the narrowest region of the microchannel 11. An inner diameter of the narrowest region of the microchannel 11 is greater than the cell diameter and less than twice of the cell diameter to prevent the cell from being directly extruded by the microchannel 11 when passing through the narrowest region, and only a single cell is allowed to pass through the narrowest region each time. The fourth section S4 is a jet stream region. The cells enter the jet stream region after passing through the narrowest region. The cell inlet 16 is arranged on one side of the first section S1, the cell outlet 17 is arranged on one side of the fourth section S4 far away from the cell inlet 16, and the jet stream inlet in2 is arranged on a conduit wall of the fourth section S4. An inner diameter of the microchannel 11 is relatively small throughout and is of the same order of magnitude as a size of the cell 28.

The mechanical monitoring device 120 includes a camera device 121 and a processor 122, and the camera device 121 is connected to the processor 122. The camera device 121 is arranged on a peripheral side of the second segment S2, and is configured to collect a cell image after cell deformation, so that the processor 122 can process the cell image to obtain the mechanical modulus of a cell.

The jet stream apparatus 130 includes a jet stream nozzle hole 131, and the jet stream nozzle hole 131 is opposite to the jet stream inlet in2 on the microchannel 11 and can be arranged coaxially. The jet stream apparatus 130 is used to provide a jet stream to perforate the cells to allow transport of a substance across the membrane.

After entering the microchannel 11 through the cell inlet 16, the cell 28 is subjected to extrusion deformation at the second section S2, the camera device 121 collects a cell image of the deformed cell 29, so that the processor 122 processes the cell image to obtain the mechanical modulus of a cell, and the jet stream intensity of the jet stream apparatus 130 for perforating the cell 29 is adjusted to make the cell membrane tension of the cell 29 between the perforation threshold and the rupture threshold; in this way, insufficient cell perforation and cell rupture can be avoided, and the cell transduction efficiency and the cell survival rate are improved.

It should be noted that the zero-mass jet stream-based cell introduction microfluidic system is an integrated mechanical measurement and perforation apparatus, and if no jet stream operation is performed, the device is a simple mechanical measurement apparatus.

The above briefly describes the zero-mass jet stream-based cell introduction microfluidic system, and the microchannel-based jet stream control method is described in detail below.

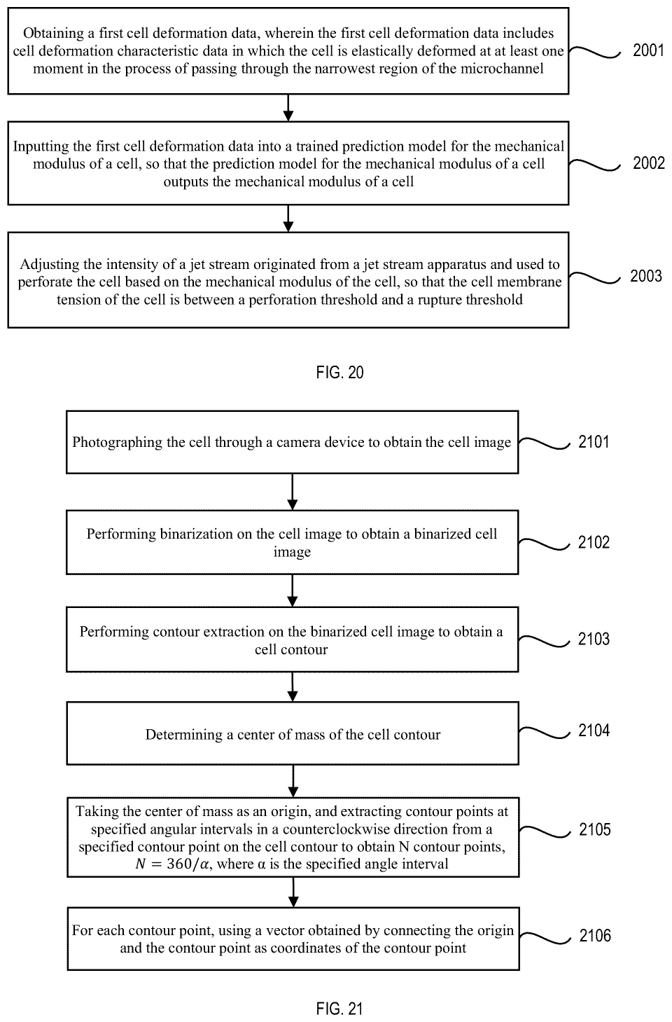

FIG. 20 is a flowchart of a microchannel-based jet stream control method according to an exemplary embodiment. Referring to FIG. 20, the microchannel-based jet stream control method may include the following steps:
Step 2001: obtaining a first cell deformation data, where the first cell deformation data includes cell deformation characteristic data in which the cell is elastically deformed at one or more moments in the process of passing through the narrowest region of the microchannel.

In this embodiment, an inner diameter of the narrowest region of the microchannel 11 is greater than the cell diameter and less than twice of the cell diameter, so that the cell is prevented from being directly extruded by the microchannel when passing through the microchannel, and only a single cell is allowed to pass through the narrowest region each time. In this way, only one cell can be collected in each cell image collected by the camera device 121, which facilitates processing by the processor 122 and improves processing efficiency.

Preferably, the inner diameter of the narrowest region of the microchannel 11 is greater than the cell diameter and less than 1.5 times the cell diameter. In this way, it is better achieved that only a single cell is allowed to pass through the narrowest region each time, that is, only one cell is present at any one position within the narrowest region at the same moment.

In this embodiment, a pressure on a side of the second section S2 close to the cell inlet 16 is greater than that on a side close to the cell outlet 17, so that the cells can flow out of the second section S2. After entering the second section S2, the undeformed cells 28 are elastically deformed due to being extruded.

In this embodiment, as shown in FIG. 9, the cells are elastically deformed to different degrees at different moments when passing through the narrowest region of the microchannel 11. For example, at a first moment t₁, the cell is not elastically deformed, and a front view P11 and a top view P12 of the cell are both circular; at a second moment t₂, a third moment t₃ and a fourth moment t₄, the cell is elastically deformed to varying degrees, a front view P21 and a top view P22 of the cell at the second moment t₂, a front view P31 and a top view P32 of the cell at the third moment t₃, and a front view P41 and a top view P42 of the cell at the fourth moment t₄ are all irregular.

In this embodiment, the first cell deformation data includes cell deformation characteristic data in which the cell is elastically deformed at a plurality of moments in the process of passing through the narrowest region of the microchannel 11. In other embodiments, the first cell deformation data may only include cell deformation characteristic data in which the cell is elastically deformed at a certain moment in the process of passing through the narrowest region of the microchannel 11.

In this embodiment, the cell deformation characteristic data is cell contour data, and the first cell deformation data further includes pressure difference information between an inlet of a variable cross-section region and an outlet of the variable cross-section region or flow information of the narrowest region, where the inlet of the narrowest region is positioned at a side of the second section S2 close to the cell inlet 16. The cell contour data is coordinates of contour points on the cell contour in the cell image. The cell image includes a front view and a top view of the cell. For each moment, the cell contour data includes first contour data of a front view and second contour data of a top view of the cell. In other embodiments, the cell image may include only a front view or a top view of the cell.

In this embodiment, the cell contour data is coordinates of 180 contour points on the cell contour in the cell image. 180 contour points are evenly distributed over the cell contour. In other embodiments, the number of contour points may be other values, which is not limited to 180 in this embodiment.

In this embodiment, as shown in FIG. 21, for each cell image, the method of obtaining cell contour data may include the following steps:
Step 2101: photographing the cell through a camera device to obtain the cell image.
Step 2101 is similar to step 1001 described above and is not described herein again.
Step 2102: performing binarization on the cell image to obtain a binarized cell image.
Step 2102 is similar to step 1002 described above and is not described herein again.
Step 2103: performing contour extraction on the binarized cell image to obtain a cell contour.
Step 2103 is similar to step 1003 described above and is not described herein again.
Step 2104: determining a center of mass of the cell contour.
Step 2104 is similar to step 1004 described above and is not described herein again.
Step 2105: taking the center of mass as an origin, and extracting contour points at specified angular intervals in a counterclockwise direction from a specified contour point on the cell contour to obtain N contour points, N=360/a, where a is the specified angle interval.
Step 2105 is similar to step 1005 described above and is not described herein again.
Step 2106: for each contour point, using a vector obtained by connecting the origin and the contour point as coordinates of the contour point.
Step 2106 is similar to step 1006 described above and is not described herein again.

The method of obtaining cell contour data is described above, and both the first contour data of the front view and the second contour data of the top view of the cell can be obtained by the above method. Details are not described herein.

Step 2002: inputting the first cell deformation data into a trained prediction model for the mechanical modulus of a cell, so that the prediction model for the mechanical modulus of a cell outputs the mechanical modulus of the cell.

Step 2002 is similar to step 702 described above and is not described herein again.

The method for predicting the mechanical modulus of a cell by using the trained prediction model for the mechanical modulus of a cell is described above. The following describes a method of training a prediction model for the mechanical modulus of a cell.

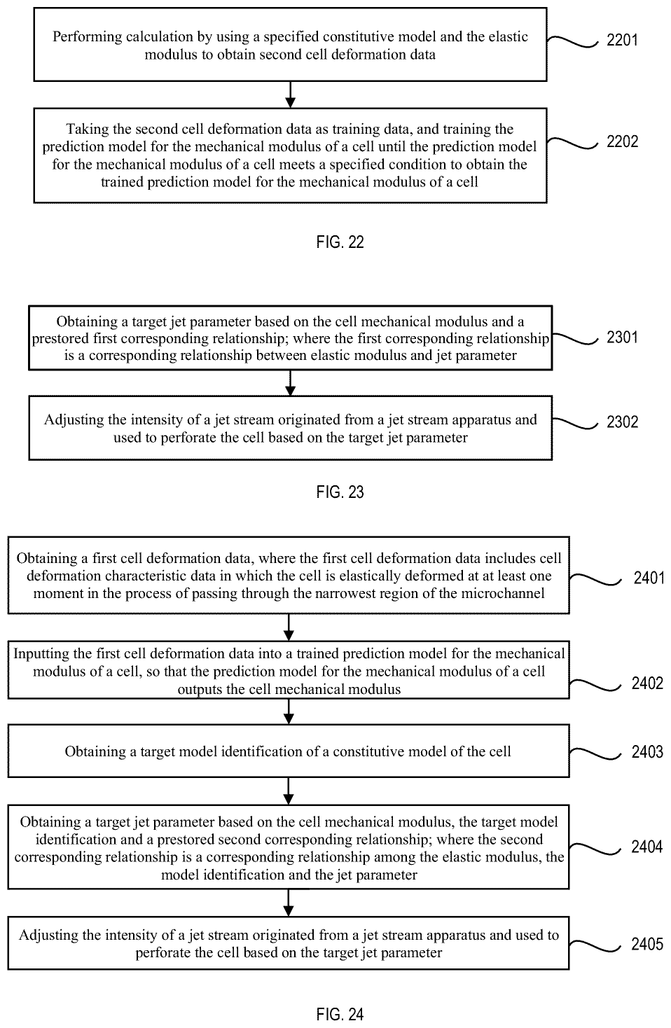

As shown in FIG. 22, the method of training a prediction model for the mechanical modulus of a cell may include the following steps:
Step 2201: performing calculation by using a specified constitutive model and the elastic modulus to obtain second cell deformation data; and
Step 2201 is similar to step 1601 described above and is not described herein again.
Step 2202: taking the second cell deformation data as training data, and training the prediction model for the mechanical modulus of a cell until the prediction model for the mechanical modulus of a cell meets a specified condition to obtain the trained prediction model for the mechanical modulus of a cell.
Step 2202 is similar to step 1602 described above and is not described herein again.

The training data is obtained by calculation, so that massive training data can be quickly obtained, the training speed is improved, and the training cost is saved. Of course, the training data may also be experimental data.

In this embodiment, the first cell deformation data is obtained and is input into the trained prediction model for the mechanical modulus of a cell, so that the prediction model for the mechanical modulus of a cell outputs the mechanical modulus of the cell; in this way, this method is faster than a method for measuring the mechanical modulus of a cell by using a measuring tool in a laboratory, and has high cell throughput, and since no measuring tool is used to apply force to the cells, the cells can be prevented from being damaged. Therefore, the technical solution provided by the present disclosure can improve the cell throughput and reduce the damage to the cells.

In other embodiments, the cell deformation characteristic data may further include any one or any combination of cell contour data, extension index, out-of-roundness, curvature ratio, relative cell size and reciprocal transit time. For example, in an embodiment, the cell deformation characteristic data may include cell contour data and an extension index. In another embodiment, the cell deformation characteristic data may include relative cell size.

In an embodiment, a calculation formula for the extension index is as follows:

$$D 1 = ( W - H ) / ( W + H )$$

where D1 is the extension index, W is the maximum length of the cell after deformation, and H is the minimum length of the cell after deformation.

In another embodiment, a calculation formula for the extension index is as follows:

$$D 1 = \frac{W}{H}$$

where W is the maximum length of the cell after deformation, and H is the minimum length of the cell after deformation.

In another embodiment, a calculation formula for the extension index is as follows:

$$D 1 = \frac{{D}_{t}}{{D}_{t + 1}} = \frac{{W}_{t} / {H}_{t}}{{W}_{t + 1} / {H}_{t + 1}}$$

where Dₜ is the deformation of the cell at the t moment, Dₜ₊₁ is the deformation of the cell at the t+1 moment, Wₜ is the maximum length of the cell after deformation at the t moment, Hₜ is the minimum length of the cell after deformation at the t moment, Wₜ₊₁ t+1 is the maximum length of the cell after deformation at the t+1 moment, and Hₜ₊₁ is the minimum length of the cell after deformation at the t+1 moment.

In another embodiment, a calculation formula for the extension index is as follows:

$$D 1 = W / 2 {R}_{1}$$

where R₁ is a radius of the cell without deformation, and W is the maximum length of the cell after deformation.

A calculation formula for out-of-roundness is as follows:

$$O = 1 - c \\ c = \frac{2 \sqrt{A π}}{p}$$

where O is out-of-roundness, c is roundness, A is an area of a cross-section of the cell after deformation, and p is a perimeter of the cross-section of the cell after deformation.

A calculation formula for curvature ratio is as follows:

$$ratio = {κ}_{min} / {κ}_{max}$$

where ratio is a curvature ratio, κₘᵢₙ is a minimum curvature of a cross-section of the cell after deformation, and κₘₐₓ is a maximum curvature of a cross-section of the cell after deformation.

A calculation formula for relative cell size is as follows:

$$D 2 = {R}_{1} / {R}_{2}$$

where D2 is the relative cell size, R₁ is a radius of the cell without deformation, and R₂ is a radius of an inner wall cross-section of the second section S2.

A calculation formula for reciprocal transit time is as follows:

$$D 3 = \frac{1}{Passage time}$$

where D3 is the reciprocal transit time, and Passage time is a time period for the cell to pass through the second section S2.

Step 2003: adjusting the intensity of a jet stream originated from a jet stream apparatus and used to perforate the cell based on the mechanical modulus of the cell, so that the cell membrane tension of the cell is between a perforation threshold and a rupture threshold, and the jet stream apparatus is positioned in the jet stream region of the microchannel.

In this embodiment, the cells enter the jet stream region after passing through the narrowest region, and the jet stream apparatus provides a jet stream to perforate the cell. For each set of cell mechanical moduli, there is a corresponding optimal jet stream intensity. For each optimal jet stream intensity, there is a corresponding set of jet stream parameters. Therefore, for each set of cell mechanical moduli, there is a corresponding set of jet stream parameters with which the jet stream apparatus can provide an optimal jet stream intensity, so that the cell membrane tension of the cell is between the perforation threshold and the rupture threshold. In this way, insufficient cell perforation and cell rupture can be avoided, and the cell can be perforated fully, which not only improves the cell transduction efficiency, but also increases the cell survival rate.

In this embodiment, the jet stream intensity of the jet stream apparatus for perforating the cell is adjusted by adjusting the jet stream parameter of the jet stream apparatus. The jet stream parameter includes a jet stream frequency and a jet stream amplitude.

In this embodiment, as shown in FIG. 23, the step 2003 may include the following steps:
Step 2301: obtaining a target jet stream parameter based on the mechanical modulus of the cell and a prestored first corresponding relationship; where the first corresponding relationship is a corresponding relationship between elastic modulus and jet stream parameter; and
Step 2302: adjusting the jet stream intensity of the jet stream apparatus for perforating the cell based on the target jet stream parameter.

In this embodiment, the constitutive model is known, and only the corresponding relationship between the elastic modulus and the jet stream parameter may be prestored. After the mechanical modulus of a cell is obtained, the first corresponding relationship can be inquired based on the mechanical modulus of the cell, and the corresponding target jet stream parameter can be obtained, where the target jet stream parameter includes a target jet stream amplitude, a target jet stream frequency and pressure difference information or flow information between one side of the jet stream region close to the cell inlet of the microchannel and one side of the cell outlet of the microchannel, so that the inquiring logic is simple, and the efficiency is high.

In this embodiment, first cell deformation data is obtained, and the first cell deformation data is input into the trained prediction model for the mechanical modulus of a cell, so that the prediction model for the mechanical modulus of a cell outputs the mechanical modulus of the cell, and then the jet stream intensity of the jet stream apparatus for perforating the cell is adjusted based on the mechanical modulus of the cell, so that the cell membrane tension of the cell is between the perforation threshold and the rupture threshold. In this way, insufficient cell perforation and cell rupture can be avoided, and the cell can be perforated fully, which not only improves the cell transduction efficiency, but also increases the cell survival rate.

In addition, the mechanical modulus of a cell is measured during the movement of cell in the microchannel and before entering the jet stream region, the data timeliness is strong, and the data is more accurate, so that the jet stream intensity of the jet stream apparatus for perforating the cell can be more accurately adjusted.

Furthermore, the microchannel-based jet stream control method integrates the measurement of the mechanical modulus of a cell and the adjustment of the jet stream intensity, so that the process is tightly connected, the time can be greatly saved, and the transmembrane transduction efficiency can be improved.

FIG. 24 is a flowchart of a microchannel-based jet stream control method according to another exemplary embodiment. Referring to FIG. 24, the microchannel-based jet stream control method may include the following steps:
Step 2401: obtaining a first cell deformation data, where the first cell deformation data includes cell deformation characteristic data in which the cell is elastically deformed at one or more moments in the process of passing through the narrowest region of the microchannel.
Step 2402: inputting the first cell deformation data into a trained prediction model for the mechanical modulus of a cell, so that the prediction model for the mechanical modulus of a cell outputs the mechanical modulus of the cell.

In this embodiment, steps 2401 to 2402 are similar to steps 2001 to 2002 and are not described herein again.
Step 2403: obtaining a target model identification of a constitutive model of the cell.

In this embodiment, the processor 122 may provide a plurality of prediction model for the mechanical modulus of a cells, wherein each prediction model for the mechanical modulus of a cell is used to predict the mechanical modulus of a cell of the corresponding constitutive model. Therefore, a corresponding target jet stream parameter is determined based on the elastic modulus and a target model identification of the constitutive model.

In this embodiment, the prediction model for the mechanical modulus of a cells and the constitutive models are in a one-to-one correspondence, and the identification of the prediction model for the mechanical modulus of a cell may be used as the identification of the corresponding constitutive model. Therefore, in this embodiment, a target model identification of the constitutive model of the cell can be obtained by obtaining the identification of the prediction model for the mechanical modulus of a cell.

Of course, obtaining the target model identification of the constitutive model of the cell may be implemented in another manner, for example, by receiving a target model identification of a constitutive model of a cell input by a user.

Step 2404: obtaining a target jet stream parameter based on the mechanical modulus of the cell, the target model identification and a prestored second corresponding relationship; where the second corresponding relationship is a corresponding relationship among the elastic modulus, the model identification and the jet stream parameter.

In this embodiment, the corresponding relationship among the elastic modulus, the model identification and the jet stream parameter is prestored. After the target model identification and the mechanical modulus of a cell of the constitutive model of the cell are obtained, the second corresponding relationship can be inquired based on the mechanical modulus of the cell and the target model identification, and the target jet stream parameter is obtained.

Step 2405: adjusting the jet stream intensity of the jet stream apparatus for perforating the cell based on the target jet stream parameter.

In this embodiment, in a scenario where constitutive models of a plurality of cells can be used, after switching the constitutive model of the cell, the target model identification of the constitutive model and the mechanical modulus of a cell of the cell may be obtained first, and then a second corresponding relationship is queried based on the mechanical modulus of the cell and the target model identification, so as to obtain a target jet stream parameter. Therefore, even if the constitutive model of the cell is switched, the jet stream intensity of the jet stream apparatus for perforating the cell can be conveniently adjusted.

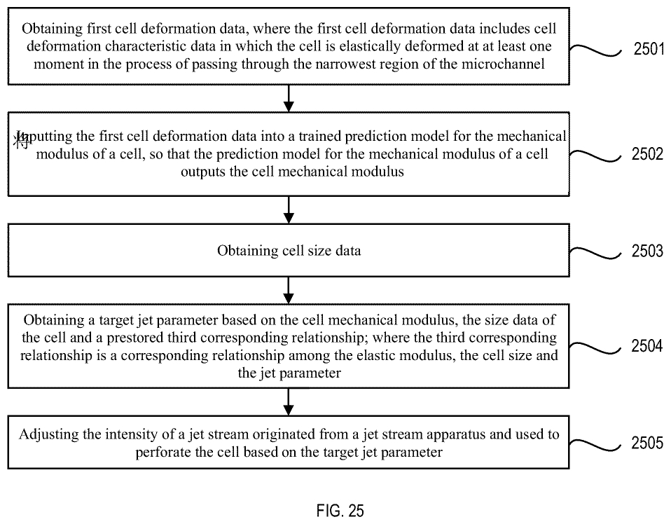

Another exemplary embodiment of the present disclosure further provides a microchannel-based jet stream control method. As shown in FIG. 25, the microchannel-based jet stream control method may include the following steps:
Step 2501: obtaining a first cell deformation data, where the first cell deformation data includes cell deformation characteristic data in which the cell is elastically deformed at one or more moments in the process of passing through the narrowest region of the microchannel.
Step 2502: inputting the first cell deformation data into a trained prediction model for the mechanical modulus of a cell, so that the prediction model for the mechanical modulus of a cell outputs the mechanical modulus of the cell.

In this embodiment, steps 2501 to 2502 are similar to steps 2001 to 2002 and are not described herein again.

Step 2503: obtaining size data of the cell.

In this embodiment, the cell size is different, and the optimal jet stream intensity of the jet stream apparatus for perforating the cell is also different. Therefore, it is also necessary to determine the target jet stream parameter based on size data of the cell.

In this embodiment, the cell may be photographed by a camera device, a cell image is obtained, and then size data of the cell may be obtained based on the cell image. In this way, the obtained size data of the cell is more targeted, so that the jet stream intensity for perforating the cell is adjusted to be more targeted, and the jet stream intensity of each cell can be precisely controlled.

In an embodiment, the camera device can photograph the undeformed cell to obtain a front view as the cell image, and then the size data of the cell is obtained based on the cell image. In this way, the data processing is simpler, the speed is higher, and the efficiency can be improved. Of course, other solutions may be used to obtain the size data of the cell, which is not limited to the solution provided by the present disclosure for obtaining the size data of the cell.

Step 2504: obtaining a target jet stream parameter based on the mechanical modulus of the cell, the size data of the cell and a prestored third corresponding relationship; where the third corresponding relationship is a corresponding relationship among the elastic modulus, the cell size and the jet stream parameter.

In this embodiment, the corresponding relationship among the elastic modulus, the model identification and the jet stream parameter is prestored. After the target model identification and the mechanical modulus of a cell of the constitutive model of the cell are obtained, the second corresponding relationship can be inquired based on the mechanical modulus of the cell and the target model identification, and the target jet stream parameter is obtained.

Step 2505: adjusting the jet stream intensity of the jet stream apparatus for perforating the cell based on the target jet stream parameter.

In this embodiment, the size data and the mechanical modulus of a cell may be obtained first, and then the second corresponding relationship is queried based on the mechanical modulus of the cell and the target model identification, so as to obtain a target jet stream parameter. Therefore, even if the constitutive model of the cell is switched, the jet stream intensity of the jet stream apparatus for perforating the cell can be conveniently adjusted.

In this embodiment, the target jet stream parameter can be obtained based on the mechanical modulus of the cell, the size data of the cell and a prestored third corresponding relationship, so as to adjust the jet stream intensity of the jet stream apparatus for perforating the cell. Therefore, even if the mechanical modulus of a cell and the cell size are changed, the jet stream intensity of the jet stream apparatus for perforating the cell can be conveniently adjusted. In addition, in this embodiment, the jet stream intensity of the jet stream apparatus for perforating the cell can be adjusted in a more targeted manner, and the jet stream intensity of each cell can be controlled more precisely.

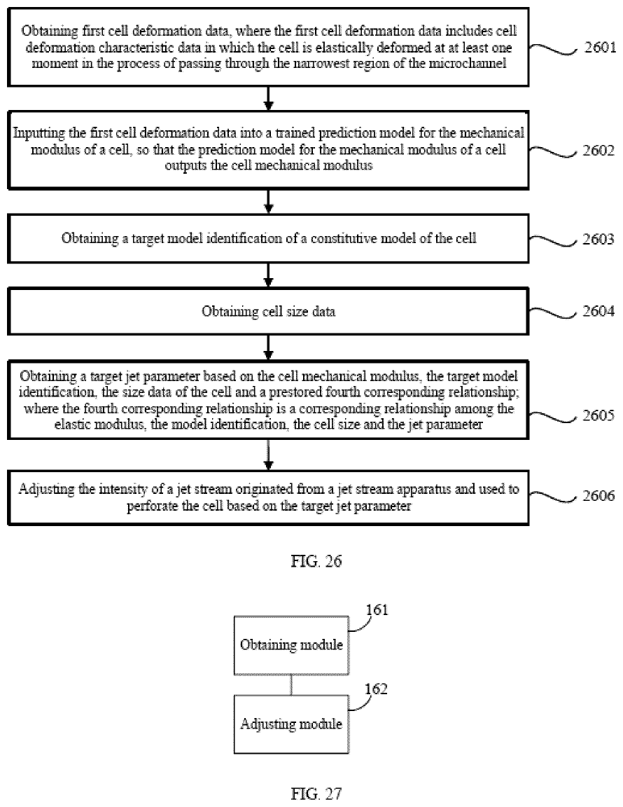

Another exemplary embodiment of the present disclosure further provides a microchannel-based jet stream control method. As shown in FIG. 26, the microchannel-based jet stream control method may include the following steps:
Step 2601: obtaining a first cell deformation data, where the first cell deformation data includes cell deformation characteristic data in which the cell is elastically deformed at at least one moment in the process of passing through the narrowest region of the microchannel.
Step 2602: inputting the first cell deformation data into a trained prediction model for the mechanical modulus of a cell, so that the prediction model for the mechanical modulus of a cell outputs the mechanical modulus of the cell.

In this embodiment, steps 2601 to 2602 are similar to steps 2001 to 2002 and are not described herein again.

Step 2603: obtaining a target model identification of a constitutive model of the cell.

In this embodiment, step 2603 is similar to step 2403 and is not described herein again.

Step 2604: obtaining size data of the cell.

In this embodiment, the cell size is different, and the optimal jet stream intensity of the jet stream apparatus for perforating the cell is also different. Therefore, it is also necessary to determine the target jet stream parameter based on size data of the cell.

In this embodiment, a target category of the cell may be obtained first, and the size data of the cell may be obtained based on the target category and a fifth corresponding relationship, where the fifth corresponding relationship is a corresponding relationship between the cell category and the cell size. The target category of the cell may be obtained by receiving a target category of a cell input by a user. The target category of the cell may also be obtained by photographing the cell by a camera device, obtaining a cell image, and then obtaining the target category of the cell based on the cell image.

Step 2605: obtaining a target jet stream parameter based on the mechanical modulus of the cell, the target model identification, the size data of the cell and a prestored fourth corresponding relationship; where the fourth corresponding relationship is a corresponding relationship among the elastic modulus, the model identification, the cell size and the jet stream parameter.

In this embodiment, after the mechanical modulus of a cell, the target model identification and the size data of the cell are obtained, the fourth corresponding relationship may be queried based on the mechanical modulus of the cell, the target model identification and the size data of the cell, so as to obtain the target jet stream parameter.

Step 2606: adjusting the jet stream intensity of the jet stream apparatus for perforating the cell based on the target jet stream parameter.

In this embodiment, the target jet stream parameter may be obtained based on the mechanical modulus of the cell, the target model identification, the size data of the cell and the prestored fourth corresponding relationship, so as to adjust the jet stream intensity of the jet stream apparatus for perforating the cell. Therefore, even if the constitutive model of the cell is switched, and the cell size is changed, the jet stream intensity of the jet stream apparatus for perforating the cell can be conveniently adjusted. In addition, in this embodiment, the jet stream intensity of the jet stream apparatus for perforating the cell can be adjusted in a more targeted manner, and the jet stream intensity of each cell can be controlled more precisely.

FIG. 27 is a block diagram of a microchannel-based jet stream control apparatus according to an exemplary embodiment. As shown in FIG. 27, in this embodiment, the microchannel-based jet stream control apparatus includes:
an obtaining module 161, configured to obtain the mechanical modulus of a cell before the cell enters a jet stream region of a microchannel; and
an adjusting module 162, configured to adjust a jet stream intensity of a jet stream apparatus for perforating the cell based on the mechanical modulus of the cell, so that the cell membrane tension of the cell is between a perforation threshold and a rupture threshold, and the jet stream apparatus is positioned in the jet stream region.

In an embodiment, the microchannel has at least two different cross-sectional areas, the narrowest region of the microchannel has an inner diameter greater than a cell diameter and less than twice of the cell diameter to prevent the cell from being directly extruded by the microchannel when passing through the narrowest region, and only a single cell is allowed to pass through the narrowest region each time; and the cell enters the jet stream region after passing through the narrowest region. As shown in FIG. 28, the obtaining module 161 includes:
an obtaining submodule 1611, configured to obtain first cell deformation data, where the first cell deformation data includes cell deformation characteristic data in which the cell is elastically deformed at one or more moments in the process of passing through the narrowest region; and
a prediction submodule 1612, configured to input the first cell deformation data into a trained prediction model for the mechanical modulus of a cell, so that the prediction model for the mechanical modulus of a cell outputs the mechanical modulus of the cell.

An embodiment of the present disclosure further provides an electronic device, which includes a memory and a processor, where the memory is configured to store a computer program executable by the processor, and the processor is configured to execute the computer program in the memory to implement the method in any one of the foregoing embodiments.

An embodiment of the present disclosure further provides a computer-readable storage medium, and when an executable computer program in the storage medium is executed by a processor, the method in any one of the foregoing embodiments is implemented.

An embodiment of the present disclosure further provides a computer program product, which includes a computer program, and when the computer program is executed by a processor, the method in any one of the foregoing embodiments is implemented.

For the apparatus in the foregoing embodiment, specific manners of performing operations by the processor are described in detail in embodiments related to the method, and details are not described herein.

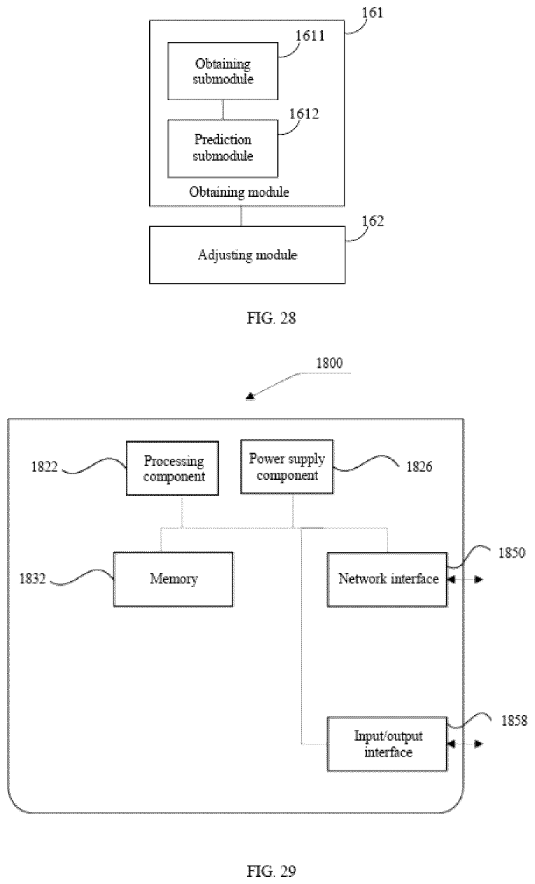

FIG. 29 is a block diagram of an electronic device according to an exemplary embodiment. For example, the electronic device 1800 may be provided as a server. Referring to FIG. 29, the device 1800 includes a processing component 1822, which further includes one or more processors, and a memory resource represented by a memory 1832, configured to store instructions, such as application programs, that may be executed by the processing components 1822. The application programs stored in the memory 1832 may include one or more modules, each corresponding to a set of instructions. In addition, the processing component 1822 is configured to execute instructions to perform the microchannel-based jet stream control method described above.

The device 1800 may also include a power supply component 1826 configured to perform power management of device 1800, a wired or wireless network interface 1850 configured to connect the device 1800 to a network, and an input/output (I/O) interface 1858. The device 1800 may operate an operating system stored in memory 1832, such as Windows Server™, MacOS X™, Unix™, Linux™, FreeBSD™, or the like.

In an exemplary embodiment, a non-transitory computer-readable storage medium including instructions, such as a memory 1832 including instructions is further provided, where the instructions may be executed by a processing component 1822 of the device 1800 to perform the method described above. For example, the non-transitory computer-readable storage medium may be a ROM, a random-access memory (RAM), a CD-ROM, a magnetic tape, a floppy disk, an optical data storage device, or the like.

In the present disclosure, the terms “first” and “second” are merely intended for description, but cannot be understood as an indication or implication of relative importance. The term “a plurality of” refers to two or more than two, unless expressly limited otherwise.

The above description of the embodiments is to facilitate those of ordinary skill in the art to understand and apply the present disclosure. It will be readily apparent to those skilled in the art that various modifications to these embodiments may be made, and the generic principles defined herein may be applied to other embodiments without inventive efforts. Therefore, the present disclosure is not limited to the embodiments herein. Improvements and modifications made by those skilled in the art based on the contents disclosed in the present disclosure without departing from the scope and spirit of the present disclosure are all within the scope of the present disclosure.

What is claimed is:

1. A microchannel-based jet stream control method, comprising:
obtaining the mechanical modulus of a cell before the cell enters a jet stream region of a microchannel (11); and
adjusting the intensity of a jet stream originated from a jet stream apparatus and used to perforate the cell based on the mechanical modulus of the cell, so that the cell membrane tension of the cell is between a perforation threshold and a rupture threshold.

2. The microchannel-based jet stream control method according to claim 1, wherein the microchannel (11) has at least two different cross-sectional areas, the narrowest region of the microchannel (11) has an inner diameter greater than a cell diameter and less than twice of the cell diameter to prevent the cell from being directly extruded by the microchannel (11) when passing through the narrowest region, and only a single cell is allowed to pass through the narrowest region each time; the cell enters the jet stream region after passing through the narrowest region; and
the obtaining the mechanical modulus of a cell before the cell enters a jet stream region of a microchannel comprises:
obtaining a first cell deformation data, wherein the first cell deformation data comprises cell deformation characteristic data in which the cell is elastically deformed at one or more moments in the process of passing through the narrowest region; and
inputting the first cell deformation data into a trained prediction model for the mechanical modulus of a cell, so that the prediction model for the mechanical modulus of a cell outputs the mechanical modulus of the cell.

3. The microchannel-based jet stream control method according to claim 2,
wherein the first cell deformation data further comprises pressure difference information between an inlet of a variable cross-section region and an outlet of the variable cross-section region or flow information of the narrowest region, the variable cross-section region is a region between a cell inlet (16) and the jet stream region of the microchannel (11), the variable cross-section region comprises the narrowest region, and a cross-sectional area of a region in the variable cross-section region positioned on two sides of the narrowest region is greater than a cross-sectional area of the narrowest region;
wherein the cell deformation characteristic data comprises at least one of cell contour data, extension index, out-of-roundness, curvature ratio, relative cell size and reciprocal transit time; when the cell deformation characteristic data comprises cell contour data, the cell contour data is coordinates of contour points on a cell contour in a cell image; and for each moment, the cell contour data comprises a first contour data of a front view and a second contour data of a top view of the cell.

4. The microchannel-based jet stream control method according to claim 2,
wherein the prediction model for the mechanical modulus of a cell is a fully connected neural network; the fully-connected neural network comprises an input layer (input), a flattening layer (flatten), a merging layer (merge), at least one hidden layer (hide1, hide2, hide3) and an output layer (output) which are connected in sequence;
wherein the input layer (input) is used to input the first cell deformation data; the flattening layer (flatten) is used to flatten the first cell deformation data to obtain a flattened first cell deformation data; the merging layer (merge) is used to merge the flattened first cell deformation data to obtain a merged data; the merging layer (merge) is fully connected to the hidden layers (hide1, hide2, hide3), the last hidden layer in the at least one hidden layer (hide1, hide2, hide3) is connected to the output layer (output), and the output layer is used to output the mechanical modulus of a cell.

5. The microchannel-based jet stream control method according to claim 3, wherein the obtaining cell deformation characteristic data comprises:
photographing the cell through a camera device to obtain the cell image;
performing binarization on the cell image to obtain a binarized cell image;
performing contour extraction on the binarized cell image to obtain a cell contour;
determining a center of mass of the cell contour;
extracting contour points from the cell contour based on a specified angle interval to obtain N contour points, N=360/a, wherein a is the specified angle interval; and
obtaining a coordinate of each of the contour points; wherein the center of mass is an origin.

6. The microchannel-based jet stream control method according to claim 2,
wherein the prediction model for the mechanical modulus of a cell is a convolutional neural network; the first cell deformation data is a cell image; and the cell image comprises a front view and/or a top view of the cell.

7. The microchannel-based jet stream control method according to claim 2,
wherein the prediction model for the mechanical modulus of a cell is trained by the following method:
performing calculation by using a specified constitutive model and the elastic modulus to obtain a second cell deformation data; and
taking the second cell deformation data as training data, and training the prediction model for the mechanical modulus of a cell until the prediction model for the mechanical modulus of a cell meets a specified condition to obtain the trained prediction model for the mechanical modulus of a cell.

8. The microchannel-based jet stream control method according to claim 7,
wherein the constitutive model considers the cell as a shell-like structural object, with viscous liquids inside and outside the cell; and the constitutive model is a Hookean model, a Mooney-Rivlin model, a Neo-Hookean model, a Skalak model or an Evans & Skalak model.

9. The microchannel-based jet stream control method according to claim 1, wherein the adjusting the intensity of a jet stream originated from a jet stream apparatus and used to perforate the cell based on the mechanical modulus of the cell comprises:
obtaining a target jet stream parameter based on the mechanical modulus of the cell and a prestored first corresponding relationship; wherein the first corresponding relationship is a corresponding relationship between the elastic modulus and the jet stream parameter; and
adjusting the intensity of a jet stream originated from a jet stream apparatus and used to perforate the cell based on the target jet stream parameter.

10. The microchannel-based jet stream control method according to claim 1,
wherein before the adjusting the intensity of a jet stream originated from a jet stream apparatus and used to perforate the cell based on the mechanical modulus of the cell, the method comprises:
obtaining a target model identification of a constitutive model of the cell;
the adjusting the intensity of a jet stream originated from a jet stream apparatus and used to perforate the cell based on the mechanical modulus of the cell comprises:
obtaining a target jet stream parameter based on the mechanical modulus of the cell, the target model identification and a prestored second corresponding relationship; wherein the second corresponding relationship is a corresponding relationship among the elastic modulus, the model identification and the jet stream parameter; and
adjusting the intensity of a jet stream originated from a jet stream apparatus and used to perforate the cell based on the target jet stream parameter.

11. The microchannel-based jet stream control method according to claim 1, wherein before the adjusting the intensity of a jet stream originated from a jet stream apparatus and used to perforate the cell based on the mechanical modulus of the cell, the method comprises:
obtaining a size data of the cell;
the adjusting the intensity of a jet stream originated from a jet stream apparatus and used to perforate the cell based on the mechanical modulus of the cell comprises:
obtaining a target jet stream parameter based on the mechanical modulus of the cell, the size data and a prestored third corresponding relationship; wherein the third corresponding relationship is a corresponding relationship among the elastic modulus, the cell size and the jet stream parameter; and
adjusting the intensity of a jet stream originated from a jet stream apparatus and used to perforate the cell based on the target jet stream parameter.

12. The microchannel-based jet stream control method according to claim 1, wherein before the adjusting the intensity of a jet stream originated from a jet stream apparatus and used to perforate the cell based on the mechanical modulus of the cell, the method comprises:
obtaining a target model identification of a constitutive model of the cell; obtaining size data of the cell;
the adjusting the intensity of a jet stream originated from a jet stream apparatus and used to perforate the cell based on the mechanical modulus of the cell comprises:
obtaining a target jet stream parameter based on the mechanical modulus of the cell, the target model identification, the size data and a prestored fourth corresponding relationship; wherein the fourth corresponding relationship is a corresponding relationship among the elastic modulus, the model identification, the cell size and the jet stream parameter;
adjusting the intensity of a jet stream originated from a jet stream apparatus and used to perforate the cell based on the target jet stream parameter.

13. The microchannel-based jet stream control method according to claim 11, wherein the obtaining a size data of the cell comprises:
photographing the cell through a camera device to obtain the cell image; and obtaining the size data of the cell based on the cell image.

14. The microchannel-based jet stream control method according to claim 11, wherein the obtaining size data of the cell comprises:
obtaining a target category of the cell; and
obtaining the size data of the cell based on the target category and a fifth corresponding relationship, wherein the fifth corresponding relationship is a corresponding relationship between the cell category and the cell size.

15. The microchannel-based jet stream control method according to claim 9, wherein the target jet stream parameter comprises a target jet stream amplitude, a target jet stream frequency, and pressure difference information or flow information between one side of the jet stream region close to a cell inlet (16) of the microchannel (11) and one side of a cell outlet (17) of the microchannel (11).

16. The microchannel-based jet stream control method according to claim 1, wherein the mechanical modulus of a cell is a cell elastic modulus, a cell shear modulus, a cell bulk modulus, or a model parameter of a cell hyperelastic model.

17. The microchannel-based jet stream control method according to claim 1, applied to a magnetically responsive membrane-based microfluidic chip for intracellular material delivery, wherein the magnetically responsive membrane-based microfluidic chip for intracellular material delivery comprises:
a chip body (10), wherein a microchannel (11) and a cavity (12) are arranged on a first face of the chip body (10), a middle part of the microchannel (11) is communicated with the cavity (12) through a transition region (13), and a necking of the transition region (13) close to the microchannel (11) end is a jet stream nozzle (14);
a magnetic material film layer (30); and
a chip base (90), wherein the chip base (90) is provided with a through hole (95), and the through hole (95) is used to make a core tip of an external alternating current coil (96) close to the magnetic material film layer (30); and
wherein a first face of the chip body (10) is bonded to a first surface of the magnetic material film layer (30) to close the microchannel (11) and the cavity (12), the chip base (90) is bonded to a second surface of the magnetic material film layer (30), the through hole (95) of the chip base (90) corresponds to a position of the cavity (12) of the chip body (10), and the magnetic material film layer (30) is vibrated under the excitation of an external magnetic field, so that a medium in the cavity (12) generates a synthetic jet stream at the jet stream nozzle (14), which acts on the cell flowing in the microchannel (11), and the cell is perforated by a momentum of the synthetic jet stream to achieve material delivery.

18. A computer-readable storage medium, having a computer program stored thereon, wherein when the executable computer program in the storage medium is executed by a processor, the method according to claim 1 can be implemented.

19. A computer program product, comprising a computer program, wherein when the computer program is executed by a processor, the method according to claim 1 is performed.
